(19) United States
(12) Patent Application Publication
(10) Pub. No.: US 2004/0058353 A1
(43) Pub. Date: Mar. 25, 2004

(54) Novel human potassium channel beta subunit

(76) Inventors: Donna M. Cusimano, Lansdale, PA (US); Richard J. Swanson, Lansdale, PA (US); Victor N. Uebele, Wyncote, PA (US)

Correspondence Address: MERCK AND CO INC, P O BOX 2000, RAHWAY, NJ, 070650907

(21) Appl. No.: 10/416,413

(22) PCT Filed:
(86) PCT No.: PCT/US01/46305

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

The present invention is directed to a novel human DNA sequences encoding Kvβ2.3, a potassium channel subunit, the protein encoded by the DNA sequences, vectors comprising the DNA sequences, host cells containing the vectors, and methods of identifying inhibitors and agonists of potassium channels containing the human Kvβ2.3 subunit.

### CROSS-REFERENCE TO RELATED APPLICATIONS

Not applicable.

### STATEMENT REGARDING FEDERALLY-SPONSORED R&D

Not applicable.

### REFERENCE TO MICROFICHE APPENDIX

Not applicable.

### FIELD OF THE INVENTION

The present invention is directed to a novel human DNA sequence encoding a potassium channel subunit, protein encoded by the DNA sequence, methods of expressing the protein in recombinant cells, and methods of identifying activators and inhibitors of potassium channels comprising the subunit.

### BACKGROUND OF THE INVENTION

Voltage-gated potassium channels form transmembrane pores that open in response to changes in cell membrane potential and selectively allow potassium ions to pass through the membrane. Voltage-gated potassium channels have been shown to be involved in maintaining cell membrane potentials and controlling the repolarization of action potentials in many cells. In particular, voltage-gated potassium channels establish the resting membrane potential and modulate the frequency and duration of action potentials in neurons, muscle cells, and secretory cells. Following depolarization, voltage-gated potassium channels open, allowing potassium efflux and thus membrane repolarization. This behavior has made voltage-gated potassium channels important targets for drug discovery in connection with a variety of diseases. As a result, many voltage-gated potassium channels have been identified and many cloned. They are distinguishable by their primary amino acid sequences, tissue-specific patterns of expression, and by electrophysiological and pharmacological properties.

Many voltage-gated potassium channels are believed to be tetramers of four pore-forming, current carrying α subunits, each of which contains six transmembrane spanning segments. The α subunits making up a tetramer may be the same (in the case of homotetramers) or may be different (in the case of heterotetramers). The membrane-spanning α subunits making up the tetramers may sometimes be associated via their intracellular domains with additional, cytoplasmic β subunits, which may alter the behavior of the α subunits. The β subunits may increase the surface expression of the α subunits or modify the biophysical properties (e.g., voltage dependence or kinetics) of the α subunits. For reviews of voltage-gated potassium channels see Robertson, 1997, Trends Pharmacol. Sci. 18:474-483; Jan & Jan, 1997, J. Physiol. 505:267-282; Catterall, 1995, Ann. Rev. Biochem. 64:493-531. For a review of potassium channel β subunits, see Pongs et al., 1999, Ann. NY Acad. Sci. 868:344-355.

Voltage-gated potassium channel α subunits appear to have an ancient evolutionary lineage, preceding the divergence of the invertebrates and vertebrates. Four subfamilies (Shaker, Shab, Shaw, and Shal; also known as Kv1, Kv2, Kv3, and Kv4 channels, respectively) were originally discovered in Drosophila and later found to be conserved in mice and humans. Each of these subfamilies contains multiple members. Each α subunit has a similar overall structure: six transmembrane spanning domains (S₁-S₆), a pore forming region located between S₅ and S₆, and intracellular amino and carboxy termini. Despite similarities in structure, these four subfamilies appear to function independently and give rise to channels having different properties when expressed in Xenopus oocytes. For example, Shal channels carry a transient (A-type) potassium current, Shaw and Shab channels carry a delayed rectifier-type current, and different members of the Shaker subfamily carry either an A-type or a delayed rectifier type current. Shaw channels show no inactivation whereas Shab and delayed rectifier type Shalker channels inactivate slowly. Certain Shaker channels inactivate very fast, due to the presence of amino terminal cytoplasmic domains that act as an “inactivation ball” by blocking the inner mouth of the channel. The voltage-sensitivities of the subfamilies differ as do their ion selectivity and pharmacologic properties (Sallcoff et al., 1992, Trends Neurosci. 15:161-166).

Potassium channel β subunits were first identified as polypeptides that co-purified with α subunits in an α-dendrotoxin-labeled complex from bovine brain (Scott et al., 1994, Proc. Natl. Acad. Sci. USA 91:1637-1641). Subsequently, several subtypes of mammalian β subunit were identified. Kvβ1 and Kvβ3 arise from the same gene by tissue-specific alternative splicing (England et al., 1995, J. Biol. Chem. 270:28531-28534). There are two reported Kvβ2 subunits, Kvβ2.1 (McCormack & McCormack, 1994, Cell 79:1133-1135; GenBank accession nos. U33429 and AF029749) and Kvβ2.2 (GenBank accession no. AF044253). Immunologic studies demonstrated that Kvβ1, Kvβ2.1, and Kvβ2.2 selectively interact with Shaker (Kv1.1, Kv1.2, Kv1.3, Kv1.5, Kv1.6) α subunits and a Shal (Kv4.2) subunit, but do not interact with Shab (Kv2.1) or Shaw (Kv3.1) subunits (Nakahira et al., 1996, J. Biol. Chem. 271:7084-7089). Another study (Rhodes et al., 1996, J. Neurosci. 17:8246-8258) reported similar, though not identical, results in that Kvβ1 and Kvβ2.1 were found to associate with the α subunits Kv1.1, Kv1.2, Kv1.4, Kv1.6, and Kv2.1 in rat brain.

β subunits can affect the function or expression of potassium channels formed by α subunits. When Kvβ1 or Kvβ3 are co-expressed with certain α subunits in Xenopus oocytes, the α subunits exhibit faster inactivation kinetics. Kvβ1 increased the rate of inactivation of the Kv1.1 α subunit by more than 100-fold; Kvβ3 increased the rate of inactivation of Kv1.4 by 4- to 7-fold, although it had no effect on Kv1.1 (Morales et al., 1995, J. Biol. Chem. 270:6272-6277). Differences in the potassium currents carried by human Kv1.5 α subunits transfected into HEK 293 and mouse L cells have been attributed to the presence of an endogenous Kvβ2.1 subunit in the L cells and the lack of such a subunit in the HEK 293 cells (Uebele et al., 1996, J. Biol. Chem. 271:2406-2412). Human Kvβ2.1 was shown to modulate the inactivation properties and increase the amplitude of potassium currents carried by Kv1.4 α subunits (McCormack et al., 1995, FEBS Lett. 370:32-36). Co-expression of Kvβ2.1 and Kv1.2 promotes N-linked glycosylation, cell surface expression, and stability of the Kv1.2 α subunits (Shi et al., 1995, Neuron 16:843-852).

It is desirable to discover other, novel potassium channel β subunits related to known subunits, especially those exhibiting restricted tissue expression. Such novel subunits would be attractive targets for drug discovery and would be valuable research tools for understanding more about ion channel biology.

### SUMMARY OF THE INTENTION

The present invention is directed to a novel isolated human DNA sequence encoding a potassium channel β subunit, Kvβ2.3. The DNA of the present invention comprises the nucleotide sequence shown as SEQ.ID.NO.: 1 as well as portions of that sequence, e.g., the coding sequence, positions 1-1146 of SEQ.ID.NO.: 1. Also provided is an isolated Kvβ2.3 protein encoded by the novel DNA sequence. The Kvβ2.3 protein comprises the amino acid sequence shown as SEQ.ID.NO.: 3 as well as fragments thereof that retain substantially the same biological activity as the human Kvβ2.3 subunit protein. Methods of expressing the Kvβ2.3 proteins in recombinant systems are provided as well as methods of identifying activators and inhibitors of potassium channels comprising the Kvβ2.3 protein.

### BRIEF DESCRIPTION OF THE DRAWINGS

FIG. 1A-C shows a DNA sequence encoding Kvβ2.3 (SEQ.ID.NO.: 1). The reverse complement of SEQ.ID.NO.: 1 is shown as the lower DNA sequence in the figure and is SEQ.ID.NO.: 2. The amino acid sequence of the Kvβ2.3 protein is shown and is SEQ.ID.NO.: 3. The taa stop codon is at position 1147-1149 of SEQ.ID.NO.: 1.

FIG. 2 shows an amino acid sequence alignment of human Kvβ2.3 (SEQ.ID.NO.: 3), human Kvβ2.1 (SEQ.ID.NO.: 4) and human Kvβ2.2 (SEQ.ID.NO.: 5).

FIG. 3A-B shows in situ hybridization localization studies of Kvβ2.3 expression in the pulvinar nuclei of the thalamus. FIG. 3A shows the results using a Kvβ2.3 antisense probe; FIG. 3B shows the results using a sense probe. The presence of light regions indicates probe hybridization.

FIG. 4 shows in situ hybridization localization studies of Kvβ2.3 expression in various regions of the thalamus.

FIG. 5A-B shows alignment of each of the exons of human Kvβ2.3 cDNA with the corresponding genomic sequences. All sequences shown are portions of SEQ.ID.NO.: 1.

### DETAILED DESCRIPTION OF THE INVENTION

For the purposes of this invention:

“Substantially free from other proteins” means at least 90%, preferably 95%, more preferably 99%, and even more preferably 99.9%, free of other proteins. Thus, a human Kvβ2.3 subunit protein preparation that is substantially free from other proteins will contain, as a percent of its total protein, no more than 10%, preferably no more than 5%, more preferably no more than 1%, and even more preferably no more than 0.1%, of proteins that are not human Kvβ2.3 subunit proteins. Whether a given human Kvβ2.3 subunit protein preparation is substantially free from other proteins can be determined by conventional techniques of assessing protein purity such as, e.g., sodium dodecyl sulfate polyacrylamide gel electrophoresis (SDS-PAGE) combined with appropriate detection methods, e.g., silver staining or immunoblotting.

“Substantially free from other nucleic acids” means at least 90%, preferably 95%, more preferably 99%, and even more preferably 99.9%, free of other nucleic acids. Thus, a human Kvβ2.3 subunit DNA preparation that is substantially free from other nucleic acids will contain, as a percent of its total nucleic acid, no more than 10%, preferably no more than 5%, more preferably no more than 1%, and even more preferably no more than 0.1%, of nucleic acids that do not encode human Kvβ2.3. Whether a given human Kvβ2.3 subunit DNA preparation is substantially free from other nucleic acids can be determined by conventional techniques of assessing nucleic acid purity such as, e.g., agarose gel electrophoresis combined with appropriate staining methods, e.g., ethidium bromide staining, or by sequencing.

A “conservative amino acid substitution” refers to the replacement of one amino acid residue by another, chemically similar, amino acid residue. Examples of such conservative substitutions are: substitution of one hydrophobic residue (isoleucine, leucine, valine, or methionine) for another; substitution of one polar residue for another polar residue of the same charge (e.g., arginine for lysine; glutamic acid for aspartic acid); substitution of one aromatic amino acid (tryptophan, tyrosine, or phenylalanine) for another.

A polypeptide has “substantially the same biological activity as the human Kvβ2.3 subunit protein” if that polypeptide is able to combine with another potassium channel subunit or subunits (e.g., an α subunit or another Kvβ subunit) so as to form a complex that constitutes a functional potassium channel where the polypeptide confers upon the complex (as compared with the other subunit or subunits alone) altered electrophysiological or pharmacological properties (e.g., altered kinetics and/or voltage dependence of activation or inactivation) that are similar to the electrophysiological or pharmacological properties that the Kvβ2.3 protein confers on the other subunit and where the polypeptide has an amino acid sequence that is at least about 50%, preferably 75%, more preferably 90%, and most preferably 97% identical to SEQ.ID.NO.: 3 when measured by such standard programs as BLAST or FASTA. Examples of other potassium channel subunits with which the polypeptide may combine are: α subunits such as Shaker (e.g., Kv1.1, Kv1.2, Kv1.3, Kv1.4, Kv1.5, Kv1.6, Kv1.7, Kv1.8 or Kv1.9), Shab (e.g., Kv2.1, Kv2.2), Shaw (e.g., Kv3.1, Kv3.2, Kv3.3, or Kv3.4 and all related splice variants), and Shal (e.g., Kv4.1, Kv4.2, or Kv4.3 and all related splice variants).

“Functional potassium channel” refers to a transmembrane protein complex comprising at least a human Kvβ2.3 subunit protein or a polypeptide that has substantially the same biological activity as the human Kvβ2.3 subunit protein and, preferably, also at least a potassium channel α subunit protein where the complex selectively allows the passage of potassium or rubidium ions across the membrane.

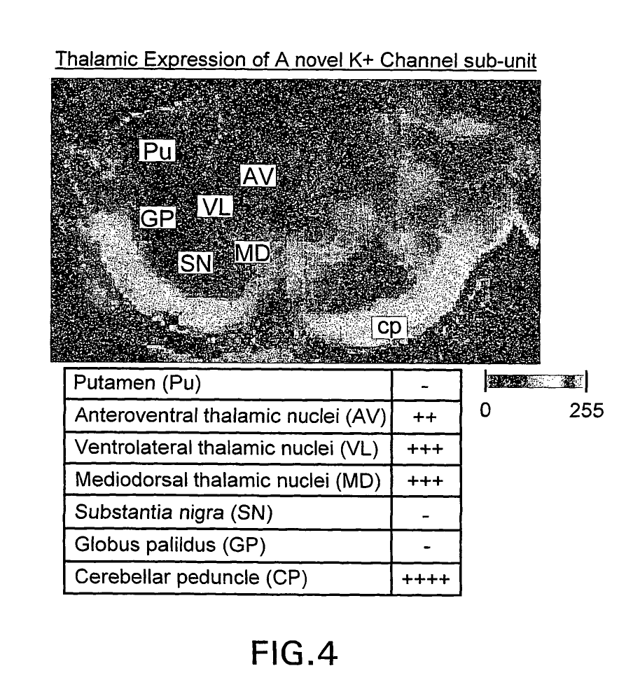

The present invention relates to the identification and cloning of DNA encoding the human Kvβ2.3 protein. A cDNA encoding Kvβ2.3 was identified from human brain mRNA by amplifying sequences homologous to other, known members of the Kvβ2 family using a RT-PCR strategy. Northern blot analysis demonstrated that human Kvβ2.3 expression is limited to the central nervous system, with the strongest expression found in the thalamus. In situ hybridization studies revealed that the major thalamic nuclei all showed robust expression. No expression was seen in the regions of the basal ganglia such as the caudate, putamen, and globus pallidus. No signal was seen in the substantia nigra. In addition to the major thalamic nuclei of the lateral mass, strong expression was noted in the pulvinar nucleus. See FIG. 4. The Table below summarizes the results of FIG. 4, with plus signs indicating expression and minus signs indicating no expression.

| | |
|---|---|
| Putamen (Pu) | − |
| Anteroventral thalamic nuclei (AV) | ++ |
| Ventrolateral thalamic nuclei (VL) | +++ |
| Mediodorsal thalamic nuclei (MD) | +++ |
| Substantia nigra (SN) | − |
| Globus pallidus (GP) | − |
| Cerebellar peduncle (CP) | ++++ |

This expression pattern suggests that modifying the activity of human Kvβ2.3 may have therapeutic relevance for sensory disorders, psychosis, and gnosis pain disorders, affective disorders, psychotic disorders, eating disorders, and neuroses).

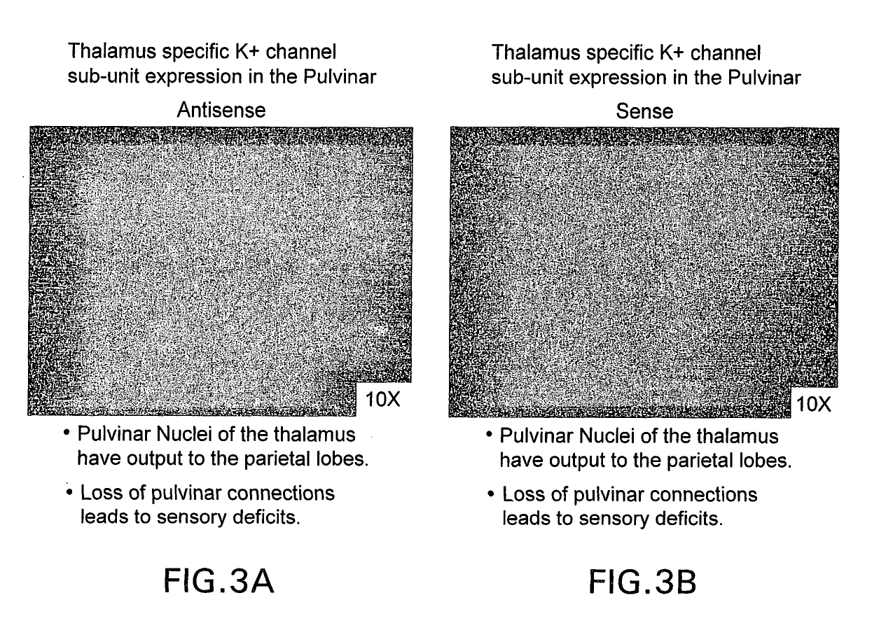

Of interest were more detailed studies of Kvβ2.3 expression in the pulvinar nuclei. FIG. 3 shows the presence of Kvβ2.3 transcripts in the pulvinar nuclei. Since the pulvinar nuclei of the thalamus have output to the parietal lobes and loss of pulvinar connections leads to sensory defects, the observed expression of Kvβ2.3 in the pulvinar nuclei suggests that agents that modulate the effects of Kvβ2.3 will be useful for the treatment of a variety of sensory defects.

The present invention provides DNAs encoding the human Kvβ2.3 subunit that are substantially free from other nucleic acids. The present invention also provides isolated and/or recombinant DNA molecules encoding the human Kvβ2.3 subunit. The present invention provides DNA molecules substantially free from other nucleic acids comprising the nucleotide sequence shown in SEQ.ID.NO.: 1.

The present invention includes isolated DNA molecules as well as DNA molecules that are substantially free from other nucleic acids comprising the coding region of SEQ.ID.NO.: 1. Accordingly, the present invention includes isolated DNA molecules and DNA molecules substantially free from other nucleic acids having a sequence comprising positions 1-1146 (the coding sequence) of SEQ.ID.NO.: 1.

Also included are recombinant DNA molecules having a nucleotide sequence comprising positions 1-1146 of SEQ.ID.NO.: 1. The novel DNA sequences of the present invention encoding the human Kvβ2.3 subunit, in whole or in part, can be linked with other DNA sequences, i.e., DNA sequences to which the human Kvβ2.3 subunit is not naturally linked, to form “recombinant DNA molecules” encoding the human Kvβ2.3 subunit. Such other sequences can include DNA sequences that control transcription or translation such as, e.g., translation initiation sequences, internal ribosome entry sites, promoters for RNA polymerase II, transcription or translation termination sequences, enhancer sequences, sequences that control replication in microorganisms, sequences that confer antibiotic resistance, or sequences that encode a polypeptide “tag” such as, e.g., a polyhistidine tract, the FLAG epitope, or the myc epitope. The novel DNA sequences of the present invention can be inserted into vectors such as plasmids, cosmids, viral vectors, P1 artificial chromosomes, or yeast artificial chromosomes.

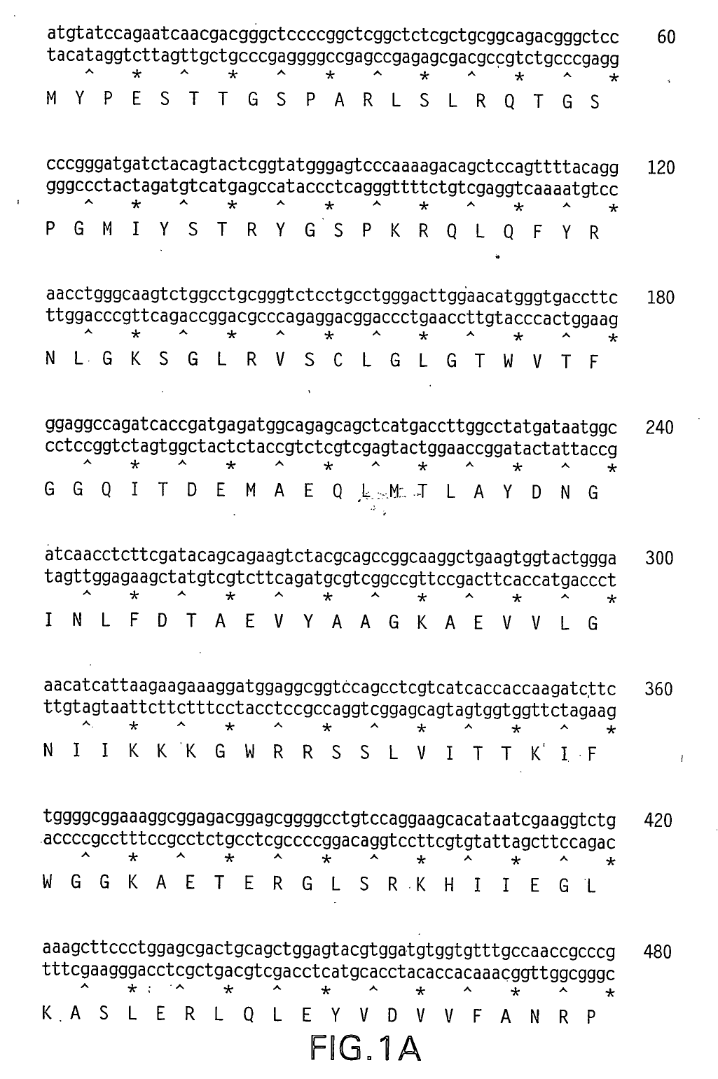

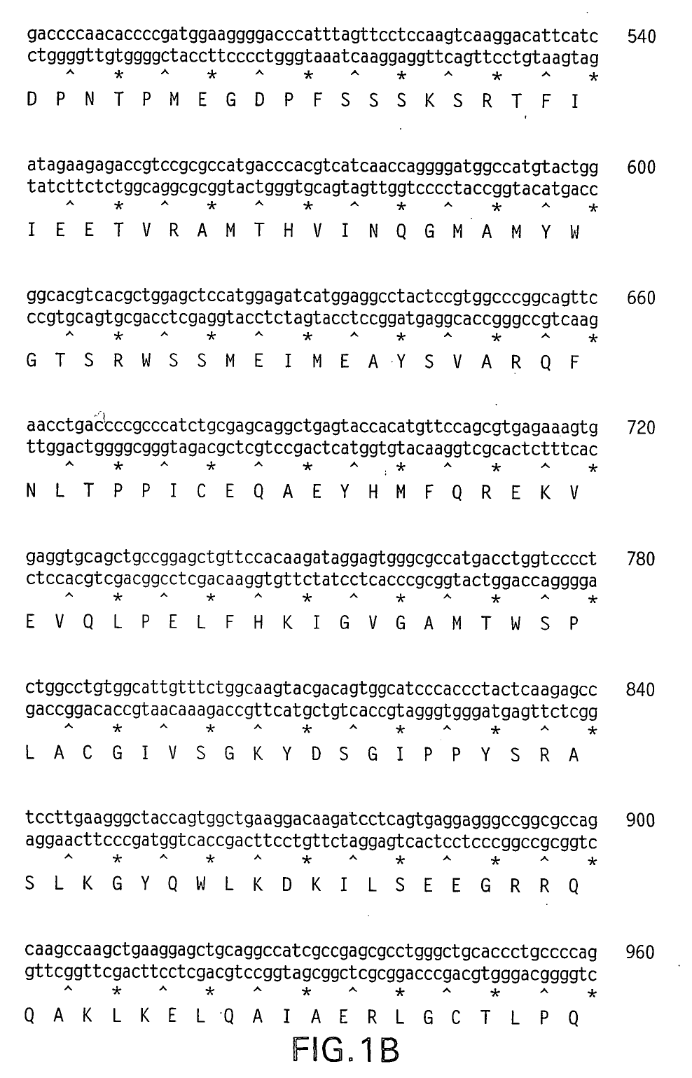

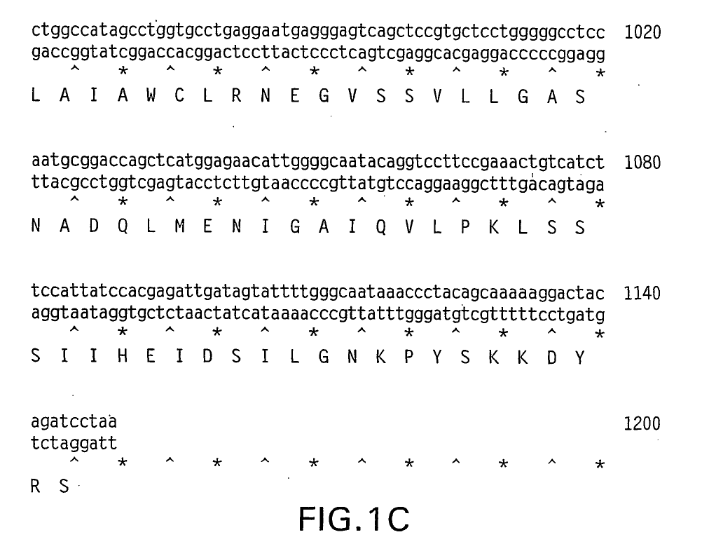

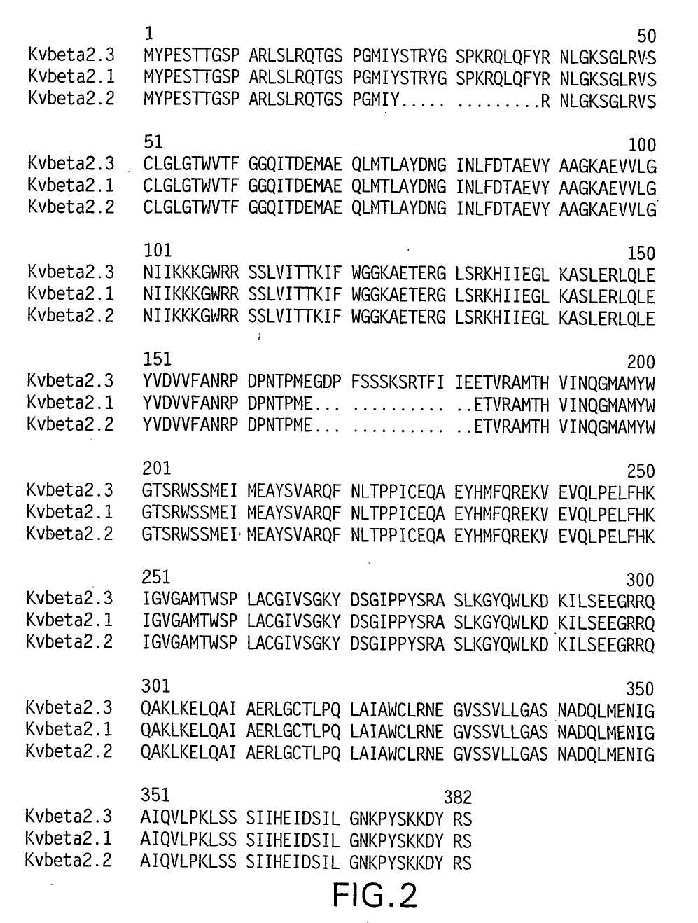

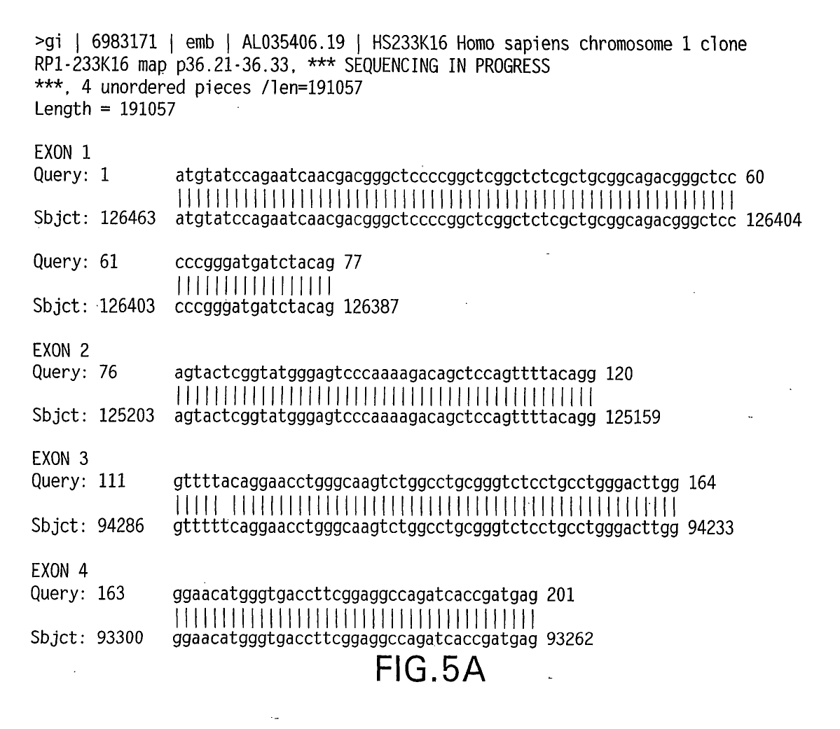

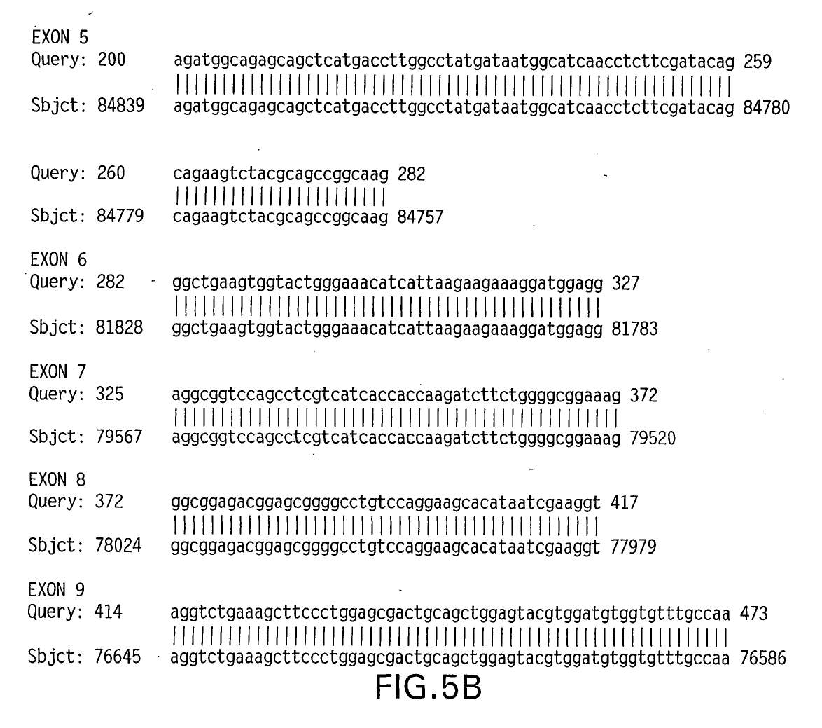

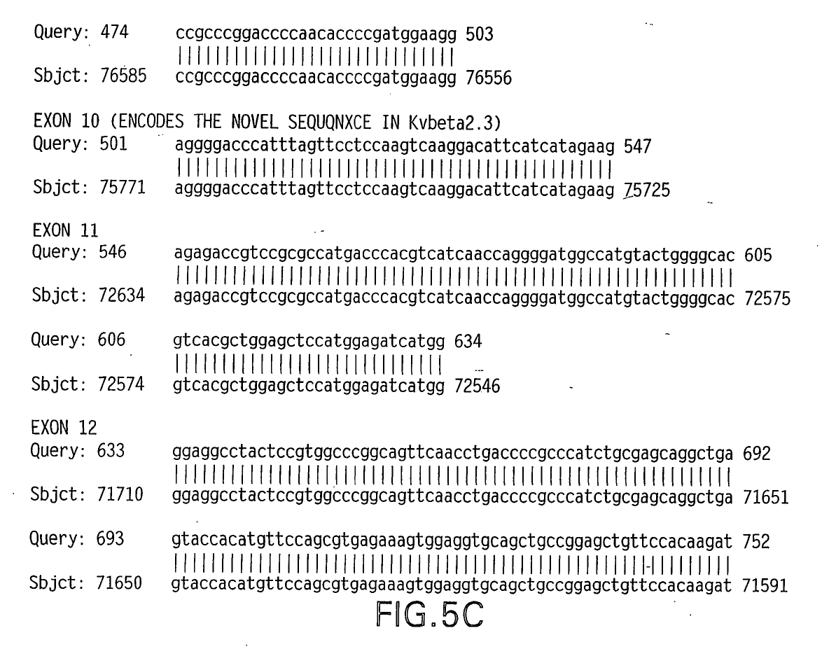

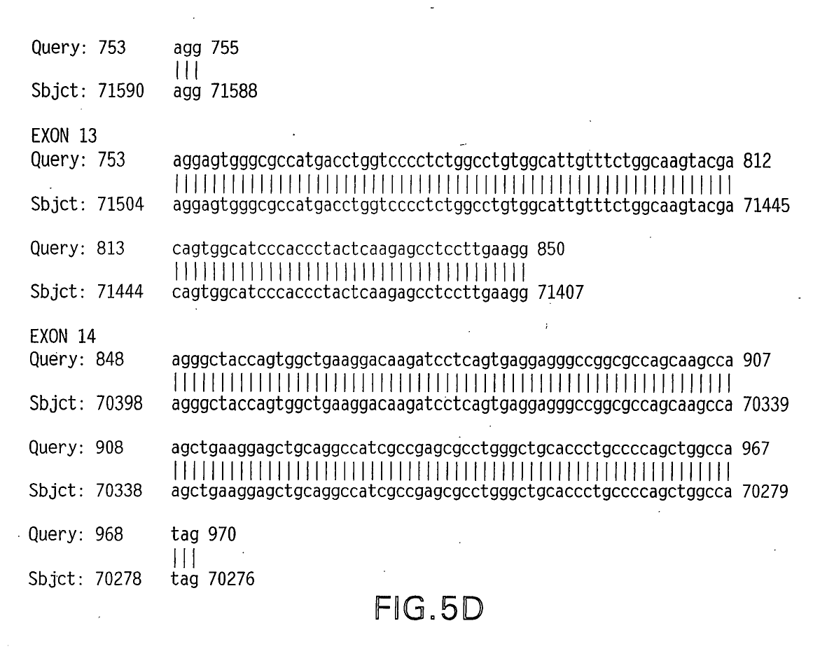

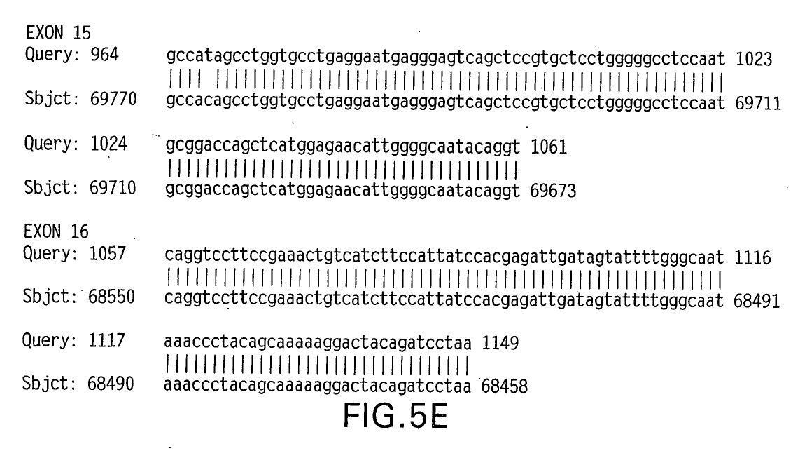

FIG. 1A-C and FIG. 2 show that the Kvβ2.3 protein contains a 15 amino acid insert (positions 167-181 of SEQ.ID.NO: 3) not found in Kvβ2.1 or Kvβ2.2. The sequence of this insert is GDPFSSSKSRTFIIE (SEQ.ID.NO: 6). This insert is encoded by a hitherto undiscovered exon, positions 502-546 of SEQ.ID.NO: 1, which has been identified in the sequence of the genomic DNA of human chromosome 1. The new sequence is encoded by the 10^{th} exon encoding the open reading frame of the Kvβ2 gene. An alignment of each of the exons with the cDNA sequence is shown in FIG. 5A-B.

Accordingly, the present invention provides a DNA sequence encoding a human Kvβ2 subunit where the DNA comprises the sequence of nucleotides 502-546 of SEQ.ID.NO: 1 and the Kvβ2 subunit binds to at least one human potassium channel α subunit selected from the group consisting of: Kv1.1, Kv1.2, Kv1.3, Kv1.4, Kv1.5, Kv1.6, Kv1.7, Kv1.8 and Kv1.9 so as to form a functional potassium channel.

Included in the present invention are DNA sequences that hybridize to the reverse complement of SEQ.ID.NO: 1 under conditions of high stringency where the hybridizing DNA sequences encode polypeptides that have substantially the same biological activity as the human Kvβ2.3 subunit protein or are capable of forming a functional potassium channel with at least one other potassium channel subunit protein. By way of example, and not limitation, a procedure using conditions of high stringency is as follows: Prehybridization of filters containing DNA is carried out for 2 hr. to overnight at 65° C. in buffer composed of 6×SSC, 5×Denhardt's solution, and 100 μg/ml denatured salmon sperm DNA. Filters are hybridized for 12 to 48 hrs at 65° C. in prehybridization mixture containing 100 μg/ml denatured salmon sperm DNA and 5-20×10⁶ cpm of ³²P-labeled probe. Washing of filters is done at 37° C. for 1 hr in a solution containing 2×SSC, 0.1% SDS. This is followed by a wash in 0.1×SSC, 0.1% SDS at 50° C. for 45 min. before autoradiography.

Other procedures using conditions of high stringency would include either a hybridization carried out in 5×SSC, 5×Denhardt's solution, 50% formamide at 42° C. for 12 to 48 hours or a washing step carried out in 0.2×SSPE, 0.2% SDS at 65° C. for 30 to 60 minutes.

Reagents mentioned in the foregoing procedures for carrying out high stringency hybridization are well known in the art. Details of the composition of these reagents can be found in, e.g., Sambrook, Fritsch, and Maniatis, 1989, Molecular Cloning: A Laboratory Manual, second edition, Cold Spring Harbor Laboratory Press. In addition to the foregoing, other conditions of high stringency which may be used are well known in the art.

The degeneracy of the genetic code is such that, for all but two amino acids, more than a single codon encodes a particular amino acid. This allows for the construction of synthetic DNA that encodes the human Kvβ2.3 subunit protein where the nucleotide sequence of the synthetic DNA differs significantly from the nucleotide sequences of SEQ.ID.NO: 1, but still encodes the same human Kvβ2.3 subunit protein as SEQ.ID.NO: 1. Such synthetic DNAs are intended to be within the scope of the present invention.

Mutated forms of SEQ.ID.NO: 1 are intended to be within the scope of the present invention. In particular, mutated forms of SEQ.ID.NO: 1 encoding a protein that, when combined with other potassium channel subunits, give rise to potassium channels having altered voltage sensitivity, current carrying properties, pharmacologic properties, or other properties as compared to potassium channels formed by combination of wild-type Kvβ2.3 protein with the other potassium channel subunit, are within the scope of the present invention. Such mutant forms can differ from SEQ.ID.NO: 1 by having nucleotide deletions, substitutions, or additions.

Also intended to be within the scope of the present invention are RNA molecules having sequences corresponding to SEQ.ID.NO: 1. Antisense oligonucleotides, DNA or RNA, that are the reverse complements of SEQ.ID.NO: 1, or portions thereof, are also within the scope of the present invention. In addition, polynucleotides based on SEQ.ID.NO: 1 in which a small number of positions are substituted with non-natural or modified nucleotides such as inosine, methyl-cytosine, or deaza-guanosine are intended to be within the scope of the present invention. Polynucleotides of the present invention can also include sequences based on SEQ.ID.NO: 1 but in which non-natural linkages between the nucleotides are present. Such non-natural linkages can be, e.g., methylphosphonates, phosphorothioates, phosphorodithionates, phosphoroarnidites, and phosphate esters. Polynucleotides of the present invention can also include sequences based on SEQ.ID.NO: 1 but having de-phospho linkages as bridges between nucleotides, e.g., siloxane, carbonate, carboxymethyl ester, acetamidate, carbamate, and thioether bridges. Other intemucleotide linkages that can be present include N-vinyl, methacryloxyethyl, methacrylarmide, or ethyleneimine linkages. Peptide nucleic acids based upon SEQ.ID.NO: 1 are also included in the present invention.

Another aspect of the present invention includes host cells that have been engineered to contain and/or express DNA sequences encoding the human Kvβ2.3 subunit protein. Such recombinant host cells can be cultured under suitable conditions to produce human Kvβ2.3 subunit protein. An expression vector containing DNA encoding the human Kvβ2.3 subunit protein can be used for the expression of the human Kvβ2.3 subunit protein in a recombinant host cell. Recombinant host cells may be prokaryotic or eukaryotic, including but not limited to, bacteria such as E. coli, fungal cells such as yeast, mammalian cells including, but not limited to, cell lines of human, bovine, porcine, monkey and rodent origin, amphibian cells such as Xenopus oocytes, and insect cells including but not limited to Drosophila and silkworm derived cell lines. Cells and cell lines which are suitable for recombinant expression of the human Kvβ2.3 subunit protein and which are widely available, include but are not limited to, L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26), MRC-5 (ATCC CCL 171), CPAE (ATCC CCL 209), Saos-2 (ATCC HTB-85), ARPE-19 human retinal pigment epithelium (ATCC CRL-2302), Xeizopzts melanophores, and Xenopus oocytes.

A variety of mammalian expression vectors can be used to express recombinant human Kvβ2.3 subunit protein in mammalian cells. Commercially available mammalian expression vectors which are suitable include, but are not limited to, pMClneo (Stratagene), pSG5 (Stratagene), pcDNAI and pcDNAIamp, pcDNA3, pcDNA3.1, pCR3.1 (Invitrogen), EBO-pSV2-neo (ATCC 37593), pBPV-1(8-2) (ATCC 37110), pdBPV-MMneo(342-12) (ATCC 37224), pRSVgpt (ATCC 37199), pRSVneo (ATCC 37198), plZD35 (ATCC 37565), and pSV2-dhfr (ATCC 37146). Another suitable vector is the PT7TS oocyte expression vector.

Following expression in recombinant cells, human Kvβ2.3 subunit protein can be purified by conventional techniques to a level that is substantially free from other proteins. Techniques that can be used include ammonium sulfate precipitation, hydrophobic or hydrophilic interaction chromatography, ion exchange chromatography, affinity chromatography, phosphocellulose chromatography, size exclusion chromatography, preparative gel electrophoresis, and alcohol precipitation. In some cases, it may be advantageous to employ protein denaturing and/or refolding steps in addition to such techniques.

Certain voltage-gated potassium channel subunits have been found to require the expression of other voltage-gated potassium channel subunits in order to be properly expressed at high levels and inserted in membranes. For example, co-expression of KCNQ3 appears to enhance the expression of KCNQ2 in Xenopus oocytes (Wang et al., 1998, Science 282:1890-1893). Also, the expression of some voltage-gated potassium channel Kv1α subunits requires other related α subunits or Kvβ2 subunits (Shi et al., 1995, Neuron 16:843-852). Accordingly, the recombinant expression of the human Kvβ2.3 subunit proteins may under certain circumstances benefit from the co-expression of other potassium channel proteins. Also, the co-expression of human Kvβ2.3 subunit protein may lead to an increase in the expression of other potassium channel subunit proteins. Therefore, the co-expression of human Kvβ2.3 and other potassium channel proteins is intended to be within the scope of the present invention.

Such co-expression can be effected by transfecting an expression vector encoding a human Kvβ2.3 subunit protein into a cell that naturally expresses another potassium channel subunit protein. Alternatively, an expression vector encoding a human Kvβ2.3 subunit protein can be transfected into a cell in which an expression vector encoding another potassium channel subunit protein has also been transfected. Preferably, such a cell does not naturally express the other potassium channel subunit protein. In preferred embodiments, the other potassium channel subunit protein is a potassium channel α subunit.

The present invention includes human Kvβ2.3 subunit proteins substantially free from other proteins. The amino acid sequence of the full-length human Kvβ2.3 subunit protein is shown in SEQ.ID.NO.: 3. Thus, the present invention includes human Kvβ2.3 subunit protein substantially free from other proteins having the amino acid sequence SEQ.ID.NO.: 3. The present invention also includes isolated human Kvβ2.3 subunit protein having the amino acid sequence SEQ.ID.NO.: 3.

Mutated forms of human Kvβ2.3 subunit protein are intended to be within the scope of the present invention. In particular, mutated forms of SEQ.ID.NO: 3 that give rise to potassium channels having altered electrophysiological or pharmacological properties when combined with other potassium channel subunits are within the scope of the present invention. Mutated forms of human Kvβ2.3 subunit protein contain amino acid substitutions, deletions, or additions as compared to SEQ.ID.NO: 3.

As with many proteins, it is possible to modify many of the amino acids of the human Kvβ2.3 subunit protein and still retain substantially the same biological activity as for the original protein. Thus, the present invention includes modified human Kvβ2.3 subunit proteins which have amino acid deletions, additions, or substitutions but that still retain substantially the same biological activity as naturally occurring human Kvβ2.3 subunit protein. It is generally accepted that single amino acid substitutions do not usually alter the biological activity of a protein (see, e.g., Molecular Biology of the Gene, Watson et al., 1987, Fourth Ed., The Benjamin/Cummings Publishing Co., Inc., page 226; and Cunningham & Wells, 1989, Science 244:1081-1085). Accordingly, the present invention includes polypeptides where one amino acid substitution has been made in SEQ.ID.NO: 3 wherein the polypeptides still retain substantially the same biological activity as naturally occurring human Kvβ2.3 subunit protein. The present invention also includes polypeptides where two or more amino acid substitutions have been made in SEQ.ID.NO: 3 wherein the polypeptides still retain substantially the same biological activity as naturally occurring human Kvβ2.3 subunit protein. In particular, the present invention includes embodiments where the above-described substitutions are conservative substitutions.

The human Kvβ2.3 subunit proteins of the present invention may contain post-translational modifications, e.g., covalently linked carbohydrate, phosphorylation, myristoylation, palmitoylation, etc.

The present invention also includes chimeric human Kvβ2.3 subunit proteins. Chimeric human Kvβ2.3 subunit proteins consist of a contiguous polypeptide sequence of at least a portion of a human Kvβ2.3 subunit protein fused to a polypeptide sequence that is not from a human Kvβ2.3 subunit protein.

The present invention also includes isolated human Kvβ2.3 subunit protein and DNA encoding the isolated subunit. Use of the term “isolated” indicates that the human Kvβ2.3 subunit protein or DNA has been removed from its normal cellular environment. Thus, an isolated human Kvβ2.3 subunit protein may be in a cell-free solution or placed in a different cellular environment from that in which it occurs naturally. The term isolated does not imply that an isolated human Kvβ2.3 subunit protein is the only protein present (although that is one of the meanings of isolated), but instead means that the isolated human Kvβ2.3 subunit protein is at least 95% free of non-amino acid material (e.g., nucleic acids, lipids, carbohydrates) naturally associated with the human Kvβ2.3 subunit protein. A human Kvβ2.3 subunit protein that is at least 90%, preferably 95%, and even more preferably 95% free of other proteins is also an isolated human Kvβ2.3 subunit protein.

It is known that certain potassium channel subunits can interact to form heteromeric complexes. For example, KCNQ2 and KCNQ3 can assemble to form a heteromeric potassium channel (Wang et al., 1998, Science 282:1890-1893). Accordingly, it is believed likely that the human Kvβ2.3 subunit protein of the present invention will also be able to form heteromeric structures with other proteins where such heteromeric structures constitute functional potassium channels. Thus, the present invention includes such heteromers comprising human Kvβ2.3 subunit protein. Preferred heteromers are those in which the human Kvβ2.3 subunit protein of the present invention forms heteromers with human potassium channel α subunits and/or Kvβ subunits.

DNA encoding the human Kvβ2.3 subunit protein can be obtained by methods well known in the art. For example, a cDNA fragment encoding full-length human Kvβ2.3 protein can be isolated from a human brain cDNA library by using the polymerase chain reaction (PCR) employing suitable primer pairs. Such primer pairs can be selected based upon the DNA sequence encoding the human Kvβ2.3 protein shown in FIG. 1A-C as SEQ.ID.NO.: 1. Suitable primer pairs would be, e.g.:

| | |
|---|---|
| 5′- ATGTATCCAGAATCAACGACG -3′ and | (SEQ.ID.NO.:7) |
| 5′- TTAGGATCTGTAGTCCTTTTTGC -3′. | (SEQ.ID.NO.:8) |

The above primers are meant to be illustrative only; one skilled in the art would readily be able to design other suitable primers based upon SEQ.ID.NO.: 1. Such primers could be produced by methods of oligonucleotide synthesis that are well known in the art.

PCR reactions can be carried out with a variety of thermostable enzymes including but not limited to AmpliTaq, AmpliTaq Gold, or Vent polymerase. For AmpliTaq, reactions can be carried out in 10 mM Tris-Cl, pH 8.3, 2.0 mM MgCl₂, 200 μM of each dNTP, 50 mM KCl, 0.2 μM of each primer, 10 ng of DNA template, 0.05 units/μl of AmpliTaq. The reactions are heated at 95° C. for 3 minutes and then cycled 35 times using suitable cycling pararneters, including, but not limited to, 95° C., 20 seconds, 62° C., 20 seconds, 72° C., 3 minutes. In addition to these conditions, a variety of suitable PCR protocols can be found in PCR Primer, A Laboratory Manual, edited by C. W. Dieffenbach and G. S. Dveksler, 1995, Cold Spring Harbor Laboratory Press; or PCR Protocols: A Guide to Methods and Applications, Michael et al., eds., 1990, Academic Press.

Since the Kvβ2.3 channel subunit of the present invention is homologous to other potassium channel β subunits (see FIG. 2), it is desirable to sequence the PCR fragments obtained by the herein-described methods, in order to verify that the desired Kvβ2.3 subunit has in fact been obtained. In particular, it is desirable to ensure that PCR fragments do not encode Kvβ2. 1 or Kvβ2.2 rather than Kvβ2.3.

By these methods, cDNA encoding the human Kvβ2.3 subunit protein can be obtained. This cDNA can be subcloned into suitable cloning vectors or expression vectors, e.g., the manmmalian expression vector pcDNA3.1 (Invitrogen, San Diego, Calif.). Human Kvβ2.3 subunit protein can then be produced by transferring expression vectors encoding the subunit or portions thereof into suitable host cells and growing the host cells under appropriate conditions. Human Kvβ2.3 subunit protein can then be isolated by methods well known in the art.

As an alternative to the above-described PCR methods, cDNA clones encoding the human Kvβ2.3 subunit protein can be isolated from cDNA libraries using as a probe oligonucleotides specific for the human Kvβ2.3 subunit and methods well known in the art for screening cDNA libraries with oligonucleotide probes. Such methods are described in, e.g., Sambrook et al., 1989, Molecular Cloning: A Laboratory Manual; Cold Spring Harbor Laboratory, Cold Spring Harbor, N.Y.; Glover, D. M. (ed.), 1985, DNA Cloning: A Practical Approach, MRL Press, Ltd., Oxford, U.K., Vol. I, II. Oligonucleotides that are specific for human Kvβ2.3 subunit protein and that can be used to screen cDNA libraries can be readily designed based upon SEQ.ID.NO.: 1, in particular nucleotides 502-546 of SEQ.ID.NO: 1. Such oligonucleotides can be synthesized by methods well-known in the art.

Genomic clones containing the human Kvβ2.3 subunit gene can be obtained by PCR using human genomic DNA as the template or from commercially available human PAC or BAC libraries available from Research Genetics, Huntsville, Ala. Alternatively, one may prepare genomic libraries, e.g., in P1 artificial chromosome vectors, from which genomic clones containing the human Kvβ2.3 subunit gene can be isolated, using probes based upon the the human Kvβ2.3 subunit DNA sequence disclosed herein. Methods of preparing such libraries are known in the art (see, e.g., Ioannou et al.,1994, Nature Genet. 6:84-89).

The novel DNA sequences of the present invention can be used in various diagnostic methods. The present invention provides diagnostic methods for determining whether a patient carries a mutation in the human Kvβ2.3 subunit gene. In broad terms, such methods comprise determining the DNA sequence of a region in or near the human Kvβ2.3 subunit gene from the patient and comparing that sequence to the sequence from the corresponding region of the human Kvβ2.3 subunit gene from a non-affected person, ie., a person who does not have the condition which is being diagnosed, where a difference in sequence between the DNA sequence of the gene from the patient and the DNA sequence of the gene from the non-affected person indicates that the patient has a mutation in the human Kvβ2.3 subunit gene.

The present invention also provides oligonucleotide probes, based upon SEQ.ID.NO: 1 that can be used in diagnostic methods to identify patients having mutated forms of the human Kvβ2.3 subunit, to determine the level of expression of RNA encoding the human Kvβ2.3 subunit, or to isolate genes homologous to the human Kvβ2.3 subunit from other species. In particular, the present invention includes DNA oligonucleotides comprising at least about 10, 15, or 18 contiguous nucleotides of SEQ.ID.NO: 1 where the oligonucleotide probe comprises no stretch of contiguous nucleotides longer than 5 from SEQ.ID.NO: 1 other than the said at least about 10, 15, or 18 contiguous nucleotides. In preferred embodiments, the oligonucleotide probes comprise at least a portion (preferably at least 10 contiguous oligonucleotides) of the sequence of nucleotides at positions 502-546 of SEQ.ID.NO: 1. The oligonucleotides can be substantially free from other nucleic acids. Also provided by the present invention are corresponding RNA oligonucleotides. The DNA or RNA oligonucleotides can be packaged in kits.

The present invention makes possible the recombinant expression of human Kvβ2.3 subunit protein in various cell types. Such recombinant expression makes possible the study of this protein so that its biochemical activity and its role in various diseases can be elucidated.

The present invention also makes possible the development of assays which measure the biological activity of potassium channels containing human Kvβ2.3 subunit protein. Such assays using recombinantly expressed human Kvβ2.3 subunit protein are especially of interest. Such assays can be used to screen libraries of compounds or other sources of compounds to identify compounds that are activators or inhibitors of the activity of potassium channels containing human Kvβ2.3 subunit protein. Such identified compounds can serve as “leads” for the development of pharmaceuticals that can be used to treat patients having diseases in which it is beneficial to enhance or suppress potassium channel activity.

In versions of the above-described assays, potassium channels containing mutant human Kvβ2.3 subunit proteins are used and inhibitors or activators of the activity of the mutant potassium channels are identified. Preferably, these inhibitors or activators do not affect potassium channels containing wild-type Kvβ2.3 in the same way as they affect potassium channels containing mutant Kvβ2.3.

Preferred cell lines for recombinant expression of human Kvβ2.3 subunit protein are those which do not express endogenous potassium channels (e.g., CV-1, NIH-3T3, CHO-K1, COS-7). Such cell lines can be loaded with ⁸⁶Rb, an ion which can pass through potassium channels. The ⁸⁶Rb-loaded cells can be exposed to collections of substances (e.g., combinatorial libraries, natural products, analogues of lead compounds produced by medicinal chemistry) and those substances that are able to alter ⁸⁶Rb efflux identified. Such substances are likely to be activators or inhibitors of potassium channels containing human Kvβ2.3 subunit protein.

Activators and inhibitors of potassium channels containing human Kvβ2.3 subunit protein are likely to be substances that are capable of binding to potassium channels containing human Kvβ2.3 subunit protein. Thus, one type of assay determines whether one or more of a collection of substances is capable of such binding.

Accordingly, the present invention provides a method for identifying substances that bind to potassium channels containing human Kvβ2.3 subunit protein comprising:

(a) providing cells expressing a potassium channel containing human Kvβ2.3 subunit protein;

(b) exposing the cells to a substance;

(c) determining the amount of binding of the substance to the cells;

(d) comparing the amount of binding in step (c) to the amount of binding of the substance to control cells where the control cells are substantially identical to the cells of step (a) except that the control cells do not express human Kvβ2.3 subunit protein;

where if the amount of binding in step (c) is greater than the amount of binding of the substance to control cells, then the substance binds to potassium channels containing human Kvβ2.3 subunit protein.

An example of control cells that are substantially identical to the cells of step (a) would be a parent cell line where the parent cell line is transfected with an expression vector encoding Kvβ2.3 protein in order to produce the cells expressing a potassium channel containing human Kvβ2.3 protein of step (a).

Another version of this assay makes use of compounds that are known to bind to potassium channels containing human Kvβ2.3 subunit protein. New binders are identified by virtue of their ability to enhance or block the binding of these known compounds. Substances that have this ability are likely themselves to be inhibitors or activators of potassium channels containing human Kvβ2.3 subunit protein.

Accordingly, the present invention includes a method of identifying substances that bind potassium channels containing human Kvβ2.3 subunit protein and thus are likely to be inhibitors or activators of potassium channels containing human Kvβ2.3 subunit protein comprising:

(a) providing cells expressing potassium channels containing human Kvβ2.3 subunit protein;

(b) exposing the cells to a compound that is known to bind to the potassium channels containing human Kvβ2.3 subunit protein in the presence and in the absence of a substance not known to bind to potassium channels containing human Kvβ2.3 subunit protein;

(c) determining the amount of binding of the compound to the cells in the presence and in the absence of the substance;

where if the amount of binding of the compound in the presence of the substance differs from the amount of binding in the absence of the substance, then the substance binds potassium channels containing human Kvβ2.3 subunit protein and is likely to be an inhibitor or activator of potassium channels containing human Kvβ2.3 subunit protein.

Generally, the known compound is labeled (e.g., radioactively, enzymatically, fluorescently, immunologically) in order to facilitate measuring its binding to the potassium channels.

The use of the words “compound” and “substance” in the above-described method is merely for the sake of differentiation. It is not meant to imply that the compound and the substance must belong to different chemical classes. For example, both the compound and the substance may be low molecular weight organic compounds of the type that are typically used as pharmaceuticals.

In the binding assays described herein, the substances identified need not bind directly to the Kvβ2.3 subunit, although that is a possibility. The substances may also bind to other subunits of the potassium channels (e.g., the α subunits). In such cases, it may be of interest to carry out control experiments to determine if the substance binds to the potassium channels when those channels do not contain the Kvβ2.3 subunit. This can be done if the cells express Kvβ2.3 because they have been transfected with an expression vector encoding Kvβ2.3. Appropriate control cells would then be the parent cells that have not been transfected with the expression vector encoding Kvβ2.3 but that express the other, α subunits of the potassium channel. In such cases, it may be that the Kvβ2.3 subunit is inducing a conformational change in the α subunits such that the substance binds the α subunits when they are in this induced conformational state but does not bind otherwise.

Once a substance has been identified by the above-described methods, it can be assayed in tests, such as those described herein, that measure the substance's effect, if any, on the biological activity of potassium channels containing the human Kvβ2.3 subunit protein, in order to determine whether the substance is an inhibitor or an activator.

In particular embodiments, the compound known to bind potassium channels containing human Kvβ2.3 subunit protein is selected from the group consisting of: α-dendrotoxin, charybdotoxin, iberiotoxin, margatoxin, mast cell degranulating peptide, hanatoxin, hongotoxin, noxiustoxin, and correolide.

The present invention includes a method of identifying activators or inhibitors of potassium channels containing human Kvβ2.3 subunit protein comprising:

(a) recombinantly expressing human Kvβ2.3 subunit protein or mutant human Kvβ2.3 subunit protein in a host cell so that the recombinantly expressed human Kvβ2.3 subunit protein or mutant human Kvβ2.3 subunit protein is incorporated into functional potassium channels with other potassium channel subunit proteins;

(b) measuring the biological activity of the functional potassium channels of step (a) in the presence and in the absence of a substance;

where a change in the biological activity of the functional potassium channels of step (a) in the presence as compared to the absence of the substance indicates that the substance is an activator or an inhibitor of potassium channels containing human Kvβ2.3 subunit protein.

It is preferred that the substances identified by the above-described method do not affect the biological activity of potassium channels that do not contain the human Kvβ2.3 subunit. This can be determined by running suitable controls, e.g., by practicing the assay with the host cells before the host cells are transfected with Kvβ2.3 so as to recombinantly express Kvβ2.3. The control cells also can be cells that have been transfected with a potassium channel β subunit other than Kvβ2.3.

It may be advantageous to recombinantly express the other subunits of potassium channels such as, e.g., α subunits. Alternatively, it may be advantageous to use host cells that endogenously express such other subunits.

In particular embodiments, the biological activity is the production or modulation of a voltage-gated potassium current or the efflux of ⁸⁶Rb. By “modulation” is meant a change in the electrophysiological characteristics of the current such as, e.g., gating voltage, activation, deactivation, or inactivation kinetics, tail current, ionic selectivity.

A preferred host cell is the Xenopus oocyte. In particular embodiments, a vector encoding human Kvβ2.3 subunit protein is transferred into Xenopus oocytes in order to cause the expression of human Kvβ2.3 subunit protein in the oocytes. Alternatively, RNA encoding human Kvβ2.3 subunit protein can be prepared in vitro and injected into the oocytes, also resulting in the expression of human Kvβ2.3 subunit protein in the oocytes. Following expression of the human Kvβ2.3 subunit protein in the oocytes, and following the formation of potassium channels containing the Kvβ2.3 subunit protein and other potassium channel subunits (which other subunits may also be transferred into the oocytes), membrane currents are measured after the transmembrane voltage is changed. A change in membrane current is observed when the potassium channels open or close, allowing or inhibiting potassium ion flow, respectively. Similar oocyte studies were reported for KCNQ2 and KCNQ3 potassium channels in Wang et al., 1998, Science 282:1890-1893 and for minK-containing channels by Goldstein & Miller, 1991, Neuron 7:403-408. These references and references cited therein can be consulted for guidance as to how to carry out such studies.

Inhibitors of potassium channels containing human Kvβ2.3 subunit protein can be identified by exposing the oocytes to substances or collections of substances and determining whether the substances can block or diminish the membrane currents observed in the absence of the substance.

Accordingly, the present invention provides a method of identifying inhibitors of potassium channels containing human Kvβ2.3 subunit protein comprising:

(a) expressing human Kvβ2.3 subunit protein in Xenopus oocytes such that potassium channels containing the human Kvβ2.3 subunit protein are formed;

(b) changing the transmembrane potential of the oocytes in the presence and the absence of a substance;

(c) measuring membrane potassium currents following step (b);

where if the membrane potassium currents measured in step (c) are greater in the absence rather than in the presence of the substance, then the substance is an inhibitor of potassium channels containing human Kvβ2.3 subunit protein.

Although Xenopus oocytes are preferred for use in the above-described method, other types of eurkaryotic cells can also be used, e.g., HEK 293 cells, HeLa cells.

The present invention also includes assays for the identification of activators and inhibitors of potassium channels containing human Kvβ2.3 subunit protein that are based upon fluorescence resonance energy transfer (FRET) between a first and a second fluorescent dye where the first dye is bound to one side, generally the exterior, of the plasma membrane of a cell expressing potassium channels containing human Kvβ2.3 subunit protein and the second dye is free to move from one face of the membrane to the other face in response to changes in membrane potential. In certain embodiments, the first dye is impenetrable to the plasma membrane of the cells and is bound predominately to the extracellular surface of the plasma membrane. The second dye is trapped within the plasma membrane but is free to diffuse within the membrane. At normal (i.e., negative) resting potentials of the membrane, the second dye is bound predominately to the inner surface of the extracellular face of the plasma membrane, thus placing the second dye in close proximity to the first dye. This close proximity allows for the generation of a large amount of FRET between the two dyes. Following membrane depolarization, the second dye moves from the extracellular face of the membrane to the intracellular face, thus increasing the distance between the dyes. This increased distance results in a decrease in FRET, with a corresponding increase in fluorescent emission derived from the first dye and a corresponding decrease in the fluorescent emission from the second dye. In this way, the amount of FRET between the two dyes can be used to measure the polarization state of the membrane. For a description of this technique, see González & Tsien, 1997, Chemistry & Biology 4:269-277. See also González & Tsien, 1995, Biophys. J. 69:1272-1280 and U.S. Pat. No. 5,661,035.

In certain embodiments, the first dye is a fluorescent lectin or a fluorescent phospholipid that acts as the fluorescent donor. Examples of such a first dye are: a coumarin-labeled phosphatidylethanolamine (e.g., N-(6-chloro-7-hydroxy-2-oxo-2H-1-benzopyran-3-benzopyran-3-carboxamidoacetyl)-dimyristoylphosphatidylethanolamine) or N-(7-nitrobenz-2-oxa-1,3-diazol-4-yl)-dimyristoylphosphatidylethanolamine); a fluorescently-labeled lectin (e.g., fluorescein-labeled wheat germ agglutinin). In certain embodiments, the second dye is an oxonol that acts as the fluorescent acceptor. Examples of such a second dye are: bis(1,3-diallkyl-2-thiobarbiturate)trimethineoxonols (e.g., bis(1,3-dihexyl-2-thiobarbiturate)trimethineoxonol) or pentamethineoxonol analogues (e.g., bis(1,3-dihexyl-2-thiobarbiturate)pentamethineoxonol; or bis(1,3-dibutyl-2thiobarbiturate)pentamethineoxonol). See González & Tsien, 1997, Chemistry & Biology 4:269-277 for methods of synthesizing various dyes suitable for use in the present invention. In certain embodiments, the assay may comprise a natural carotenoid, e.g., astaxanthin, in order to reduce photodynamic damage due to singlet oxygen.

The above described assays can be utilized to discover activators and inhibitors of potassium channels containing human Kvβ2.3 subunit protein. Such assays will generally utilize cells that express potassium channels containing human Kvβ2.3 subunit protein, e.g., by transfection with expression vectors encoding human Kvβ2.3 subunit protein and other potassium channel subunits. In such cells, increases in membrane potential (i.e., depolarizations) may open the potassium channels. This will result in potassium efflux, tending to counteract the depolarization. In other words, the cells will tend to repolarize. The presence of an inhlibitor of the potassium channel containing human Kvβ2.3 subunit protein will prevent, or diminish, this repolarization. Thus, membrane potential will tend to become more positive in the presence of inhibitors. Activators of the potassium channel will open this channel and thus tend to hyperpolarize the membrane potential. Changes in membrane potential (depolarizations and hyperpolarizations) that are caused by inhibitors and agonists of potassium channels containing Kvβ2.3 protein can be monitored by the assays using FRET described above.

Accordingly, the present invention provides a method of identifying activators of potassium channels containing human Kvβ2.3 subunit protein comprising:

(a) providing test cells comprising:

(1). an expression vector that directs the expression of human Kvβ2.3 subunit protein in the cells so that potassium channels containing human Kvβ2.3 subunit protein are formed in the cells;

(2) a first fluorescent dye, where the first dye is bound to the exterior side of the plasma membrane of the cells; and

(3) a second fluorescent dye, where the second fluorescent dye is free to move from one face of the plasma membrane of the cells to the other face in response to changes in membrane potential;

(b) exposing the test cells to a substance;

(c) measuring the amount of fluorescence resonance energy transfer (FRET) in the test cells that have been exposed to the substance;

(d) comparing the amount of FRET exhibited by the test cells that have been exposed to the substance with the amount of FRET exhibited by control cells;

where if the amount of FRET exhibited by the test cells is greater than the amount of FRET exhibited by the control cells, the substance is an activator of potassium channels containing human Kvβ2.3 subunit protein;

where the control cells are either (1) cells that are essentially the same as the test cells except that they do not comprise at least one of the items listed at (a) (1)-(3) but have been exposed to the substance; or (2) test cells that have not been exposed to the substance.

The present invention also provides a method of identifying inhibitors of potassium channels containing human Kvβ2.3 subunit protein comprising:

(a) providing test cells comprising:

(1) an expression vector that directs the expression of human Kvβ2.3 subunit protein in the cells so that potassium channels containing human Kvβ2.3 subunit proteins are formed in the cells;

(2) a first fluorescent dye, where the first dye is bound to the exterior side of the plasma membrane of the cells; and

(3) a second fluorescent dye, where the second fluorescent dye is free to move from one face of the plasma membrane of the cells to the other face in response to changes in membrane potential;

(b) exposing the test cells to a substance that is suspected of being an inhibitor of potassium channels containing human Kvβ2.3 subunit protein;

(c) measuring the amount of fluorescence resonance energy transfer (FRET) in the test cells that have been exposed to the substance;

(d) comparing the amount of FRET exhibited by the test cells that have been exposed to the substance with the amount of FRET exhibited by control cells;

where if the amount of FRET exhibited by the test cells is less than the amount of FRET exhibited by the control cells, the substance is an inhibitor of potassium channels containing human Kvβ2.3 subunit protein;

where the control cells are either (1) cells that are essentially the same as the test cells except that they do not comprise at least one of the items listed at (a) (1)-(3) but have been exposed to the substance; or (2) test cells that have not been exposed to the substance.

In a variation of the assays described above, instead of the cell's membrane potential being allowed to reach steady state on its own, the membrane potential is artificially set at a potential in which the potassium channels containing human Kvβ2.3 subunit protein are open. This can be done, e.g., by variation of the external K+ concentration in a known manner (e.g., increased concentrations of external K+). If such cells having open potassium channels containing human Kvβ2.3 subunit protein are exposed to inhibitors, the potassium channels will close, and the cells' membrane potentials will be depolarized. This depolarization can be observed as a decrease in FRET.

Accordingly, the present invention provides a method of identifying inhibitors of potassium channels containing human Kvβ2.3 subunit protein comprising:

(a) providing cells comprising:

(1) an expression vector that directs the expression of human Kvβ2.3 subunit protein in the cells so that potassium channels containing human Kvβ2.3 subunit protein are formed in the cells;

(2) a first fluorescent dye, where the first dye is bound to the exterior side of the plasma membrane of the cells; and

(3) a second fluorescent dye, where the second fluorescent dye is free to move from one face of the plasma membrane of the cells to the other face in response to changes in membrane potential;

(b) adjusting the membrane potential of the cells such that the ion channel formed by the potassium channels containing human Kvβ2.3 subunit protein is open;

(c) measuring the amount of fluorescence resonance energy transfer (FRET) in the test cells;

(d) exposing the cells to a substance;

(e) measuring the amount of FRET in the test cells that have been exposed to the substance;

where if the amount of FRET exhibited by the cells that are exposed to the substance is less than the amount of FRET exhibited by the cells that have not been exposed to the substance, then the substance is an inhibitor of potassium channels containing human Kvβ2.3 subunit protein.

In particular embodiments of the above-described method, the cells of step (a) are divided into two portions and one portion is exposed to the substance in order to generate the measurement of step (e) while the other portion is not exposed to the substance in order to generate the measurement of step (c).

In particular embodiments of the above-described methods, the expression vector is transfected into the test cells.

In particular embodiments of the above-described methods, the human Kvβ2.3 subunit protein has the amino acid sequence shown in:SEQ.ID.NO.: 3. In particular embodiments of the above-described methods, the expression vector comprises positions 1-1146 of SEQ.ID.NO.: 1.

In particular embodiments of the above-described methods, the first fluorescent dye is selected from the group consisting of: a fluorescent lectin; a fluorescent phospholipid; a coumarin-labeled phosphatidylethanolamine; N-(6-chloro-7-hydroxy-2-oxo-2H-1-benzopyran-3-carboxamidoacetyl)-dimyristoylphosphatidylethanolamine); N-(7-nitrobenz-2-oxa-1,3-diazol-4-yl)-dipalmitoylphosphatidylethanolamine); and fluorescein-labeled wheat germ agglutinin.

In particular embodiments of the above-described methods, the second fluorescent dye is selected from the group consisting of: an oxonol that acts as the fluorescent acceptor; bis(1,3-dialkyl-2-thiobarbiturate)trimethineoxonols; bis(1,3-dihexyl-2-thiobarbiturate)trimethineoxonol; bis(1,3-dialkyl-2-thiobarbiturate) quatramethineoxonols; bis(1,3-dialk-yl-2-thiobarbiturate)pentamethineoxonols; bis(1,3-dihexyl-2-thiobarbiturate)pentamethineoxonol; bis(1,3-dibutyl-2-thiobarbiturate)pentamethineoxonol); and bis(1,3-dialkyl-2-thiobarbiturate) hexamethineoxonols.

In a particular embodiments of the above-described methods, the cells are eukaryotic cells. In another embodiment, the cells are mammalian cells. In other embodiments, the cells are L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26), MRC-5 (ATCC CCL 171), Xenopus melanophores, or Xenopus oocytes.

In particular embodiments of the above-described methods, the control cells do not comprise item (a)(1) but do comprise items (a)(2) and (a)(3).

In assays to identify activators or inhibitors of potassium channels containing human Kvβ2.3 subunit protein, it may be advantageous to co-express another potassium channel subunit besides the human Kvβ2.3 subunit. In particular, it may be advantageous to co-express a potassium channel subunit such as α subunits. Preferably, this is done by co-transfecting into the cells an expression vector encoding the other subunit.

While the above-described methods are explicitly directed to testing whether “a” substance is an activator or inhibitor of potassium channels containing human Kvβ2.3 subunit protein, it will be clear to one skilled in the art that such methods can be adapted to test collections of substances, e.g., combinatorial libraries, phage display libraries, collections of natural products, to determine whether any members of such collections are activators or inhibitors of potassium channels containing human Kvβ2.3 subunit protein. Accordingly, the use of collections of substances, or individual members of such collections, as the substance in the above-described methods is within the scope of the present invention. In particular, it is envisioned that libraries that have been designed to incorporate chemical structures that are known to be associated with potassium ion channel modulation, e.g., dihydrobenzopyran libraries for potassium channel activators (International Patent Publication WO 95/30642) or biphenyl-derivative libraries for potassium channel inhibitors (International Patent Publication WO 95/04277), will be of especial interest.

The present invention includes pharmaceutical compositions comprising activators or inhibitors of potassium channels comprising human Kvβ2.3 subunit protein that have been identified by the herein-described methods. The activators or inhibitors are generally combined with pharmaceutically acceptable carriers to form pharmaceutical compositions. Examples of such carriers and methods of formulation of pharmaceutical compositions containing activators or inhibitors and carriers can be found in Remington's Pharmaceutical Sciences, 18^{th} Edition, 1990, Mack Publishing Co., Easton, Pa. To form a pharmaceutically acceptable composition suitable for effective administration, such compositions will contain a therapeutically effective amount of the activators or inhibitors.

Therapeutic or prophylactic compositions are administered to an individual in amounts sufficient to treat or prevent conditions where the activity of potassium channels containing human Kvβ2.3 subunit protein is abnormal. The effective amount can vary according to a variety of factors such as the individual's condition, weight, gender, and age. Other factors include the mode of administration. The appropriate amount can be determined by a skilled physician. Generally, an effective amount will be from about 0.01 to about 1,000, preferably from about 0.1 to about 250, and even more preferably from about 1 to about 50 mg per adult human per day.

Compositions can be used alone at appropriate dosages. Alternatively, co-administration or sequential administration of other agents can be desirable.

The compositions can be administered in a wide variety of therapeutic dosage forms in conventional vehicles for administration. For example, the compositions can be administered in such oral dosage forms as tablets, capsules (each including timed release and sustained release formulations), pills, powders, granules, elixirs, tinctures, solutions, suspensions, syrups and emulsions, or by injection. Likewise, they can also be administered in intravenous (both bolus and infusion), intraperitoneal, subcutaneous, topical with or without occlusion, or intramuscular form, all using forms well known to those of ordinary skill in the pharmaceutical arts.

Compositions can be administered in a single daily dose, or the total daily dosage can be administered in divided doses of two, three, four or more times daily. Furthermore, compositions can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

The dosage regimen utilizing the compositions is selected in accordance with a variety of factors including type, species, age, weight, sex and medical condition of the patient; the severity of the condition to be treated; the route of administration; the renal, hepatic and cardiovascular function of the patient; and the particular composition thereof employed. A physician of ordinary skill can readily determine and prescribe the effective amount of the composition required to prevent, counter or arrest the progress of the condition. Optimal precision in achieving concentrations of composition within the range that yields efficacy without toxicity requires a regimen based on the kinetics of the composition's availability to target sites. This involves a consideration of the distribution, equilibrium, and elimination of a composition.

The inhibitors and activators of potassium channels containing human Kvβ2.3 subunit protein will be useful for treating a variety of diseases involving excessive or insufficient potassium channel activity.

The inhibitors of the present invention are expected to be useful in treating a variety of conditions such as: sensory disorders, psychosis, pain disorders, affective disorders, psychotic disorders, eating disorders, and neuroses. From the crystal structure of a potassium channel beta subunit (Gulbis et al., 1999, Cell 97:943-952), it is clear that the amino acid sequence unique to Kvβ2.3 (GDPFSSSKSRTFIIE (SEQ.ID.NO: 6) is in a region of the three dimensional structure of the protein that is thought to be involved in coupling the redox potential of the cell to the activity of the channel. The inhibitors and activators of the present invention are, therefore, also expected to be useful in conditions in which it is desirable to either uncouple or enhance this redox regulation of the ion channel activity.

The present invention includes a method of modulating the biological activity of potassium channels containing the Kvβ2.3 subunit in a human host in need of such modulation comprising administering a compound that modulates the activity of a potassium channel containing the Kvβ2.3 subunit where the compound does not also modulate the biological activity of potassium channels that do not contain the Kvβ2.3 subunit.

Potassium channels containing the Kvβ2.3 subunit of the present invention are useful in conjunction with screens designed to identify activators and inhibitors of other ion channels. When screening compounds in order to identify potential pharmaceuticals that specifically interact with a target ion channel, it is necessary to ensure that the compounds identified are as specific as possible for the target ion channel. To do this, it is necessary to screen the compounds against as wide an array as possible of ion channels that are similar to the target ion channel. Thus, in order to find compounds that are potential pharmaceuticals that interact with ion channel A, it is not enough to ensure that the compounds interact with ion channel A (the “plus target”) and produce the desired pharmacological effect through ion channel A. It is also necessary to determine that the compounds do not interact with ion channels B, C, D, ect. (the “minus targets”). In general, as part of a screening program, it is important to have as many minus targets as possible (see Hodgson, 1992, Bio/Technology 10:973-980, at 980). Human Kvβ2.3 subunit protein, DNA encoding human Kvβ2.3 subunit protein, and recombinant cells that have been engineered to express human Kvβ2.3 subunit protein have utility in that they can be used as “minus targets” in screens designed to identify compounds that specifically interact with other ion channels. For example, Wang et al., 1998, Science 282:1890-1893 have shown that KCNQ2 and KCNQ3 form a heteromeric potassium ion channel know as the “M-channel.” The M-channel is an important target for drug discovery since mutations in KCNQ2 and KCNQ3 are responsible for causing epilepsy (Biervert et al., 1998, Science 279:403-406; Singh et al., 1998, Nature Genet. 18:25-29; Schroeder et al., Nature 1998, 396:687-690). A screening program designed to identify activators or inhibitors of the M-channel would benefit greatly by the use of potassium channels comprising human Kvβ2.3 subunit protein as minus targets.

The present invention also includes antibodies to the human Kvβ2.3 subunit protein. Such antibodies may be polyclonal antibodies or monoclonal antibodies. The antibodies of the present invention can be raised against the entire human Kvβ2.3 subunit protein or against suitable antigenic fragments that are coupled to suitable carriers, e.g., serum albumnin or keyhole limpet hemocyanin, by methods well known in the art. Methods of identifying suitable antigenic fragments of a protein are known in the art and can be applied to the human Kvβ2.3 subunit protein. See, e.g., Hopp & Woods, 1981, Proc. Natl. Acad. Sci. USA 78:3824-3828; and Jameson & Wolf, 1988, CABIOS (Computer Applications in the Biosciences) 4:181-186.

For the production of polyclonal antibodies, human Kvβ2.3 subunit protein or antigenic fragments, coupled to a suitable carrier, are injected on a periodic basis into an appropriate non-human host animal such as, e.g., rabbits, sheep, goats, rats, mice. The animals are bled periodically and sera obtained are tested for the presence of antibodies to the injected subunit or antigen fragment. The injections can be intramuscular, intraperitoneal, subcutaneous, and the like, and can be accompanied with adjuvant.

For the production of monoclonal antibodies, human Kvβ2.3 subunit protein or antigenic fragments, coupled to a suitable carrier, are injected into an appropriate non-human host animal as above for the production of polyclonal antibodies. In the case of monoclonal antibodies, the animal is generally a mouse. The animal's spleen cells are then immortalized, often by fusion with a myeloma cell, as described in Kohler & Milstein, 1975, Nature 256:495-497. For a description of the production of monoclonal antibodies, see Antibodies: A Laboratory Manual, Harlow & Lane, eds., Cold Spring Harbor Laboratory Press, 1988.

Gene therapy may be used to introduce human Kvβ2.3 subunit protein into the cells of target organs. Nucleotides encoding human Kvβ2.3 subunit protein can be ligated into viral vectors which mediate transfer of the nucleotides by infection of recipient cells. Suitable viral vectors include retrovirus, adenovirus, adeno-associated virus, herpes virus, vaccinia virus, lentivirus, and polio virus based vectors. Alternatively, nucleotides encoding human Kvβ2.3 subunit protein can be transferred into cells for gene therapy by non-viral techniques including receptor-mediated targeted transfer using ligand-nucleotide conjugates, lipofection, membrane fusion, or direct microinjection. These procedures and variations thereof are suitable for ex vivo as well as in vivo gene therapy. Gene therapy with human Kvβ2.3 subunit protein will be particularly useful for the treatment of diseases where it is beneficial to elevate potassium channel activity.

The present invention includes processes for cloning orthologues of human Kvβ2.3 subunit protein from non-human species. In general, such processes include preparing a pair of PCR primers or a hybridization probe based upon SEQ.ID.NO.: 1 that can be used to amplify a fragment containing the non-human Kvβ2.3 subunit (in the case of PCR) from a suitable DNA preparation or to select a cDNA or genomic clone containing the non-human Kvβ2.3 subunit from a suitable library. A preferred embodiment of this process is a process for cloning the Kvβ2.3 subunit from mouse.

By providing DNA encoding mouse Kvβ2.3 subunit, the present invention allows for the generation of an animal model of human diseases in which Kvβ2.3 subunit activity is abnormal. Such animal models can be generated by making transgenic “knockout” or “knockin” mice containing altered Kvβ2.3 subunit genes. Knockout mice can be generated in which portions of the mouse Kvβ2.3 subunit gene have been deleted. Knockin mice can be generated in which mutations that have been shown to lead to human disease are introduced into the mouse gene. Such knockout and knockin mice will be valuable tools in the study of the relationship between potassium channels and disease and will provide important model systems in which to test potential pharmaceuticals or treatments for human diseases involving potassium channels.

Accordingly, the present invention includes a method of producing a transgenic mouse comprising:

(a) designing PCR primers or an oligonucleotide probe based upon SEQ.ID.NO.: 1 for use in cloning the mouse Kvβ2.3 subunit gene or cDNA;

(b) using the PCR primers or the oligonucleotide probe to clone at least a portion of the mouse Kvβ2.3 subunit gene or cDNA, the portion being large enough to use in making a transgenic mouse;

(c) producing a transgenic mouse having at least one copy of the mouse Kvβ2.3 subunit gene altered from its native state.

Methods of producing knockout and knockin mice are well known in the art. One method involves the use of gene-targeted ES cells in the generation of gene-targeted transgenic knockout mice and is described in, e.g., Thomas et al., 1987, Cell 51:503-512, and is reviewed elsewhere (Frohman et al., 1989, Cell 56:145-147; Capecchi, 1989, Trends in Genet. 5:70-76; Baribault et al., 1989, Mol. Biol. Med. 6:481-492).

Techniques are available to inactivate or alter any genetic region to virtually any mutation desired by using targeted homologous recombination to insert specific changes into chromosomal genes. Generally, use is made of a “targeting vector,” i.e., a plasmid containing part of the genetic region it is desired to mutate. By virtue of the homology between this part of the genetic region on the plasmid and the corresponding genetic region on the chromosome, homologous recombination can be used to insert the plasmid into the genetic region, thus disrupting the genetic region. Usually, the targeting vector contains a selectable marker gene as well.

In comparison with homologous extrachromosomal recombination, which occurs at frequencies approaching 100%, homologous plasmid-chromosome recombination was originally reported to only be detected at frequencies between 10⁻⁶ and 10⁻³ (Lin et al., 1985, Proc. Natl. Acad. Sci. USA 82:1391-1395; Smithies et al., 1985, Nature 317: 230-234; Thomas et al., 1986, Cell 44:419-428). Nonhomologous plasmid-chromosome interactions are more frequent, occurring at levels 10⁵-fold (Lin et al., 1985, Proc. Natl. Acad. Sci. USA 82:1391-1395) to 10²-fold (Thomas et al., 1986, Cell 44:419-428) greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening individual clones (Kim et al., 1988, Nucleic Acids Res. 16:8887-8903; Kim et al., 1991, Gene 103:227-233). Alternatively, a positive genetic selection approach has been developed in which a marker gene is constructed which will only be active if homologous insertion occurs, allowing these recombinants to be selected directly (Sedivy et al., 1989, Proc. Natl. Acad. Sci. USA 86:227-231). One of the most powerful approaches developed for selecting homologous recombinants is the positive-negative selection (PNS) method developed for genes for which no direct selection of the alteration exists (Mansour et al., 1988, Nature 336:348-352; Capecchi, 1989, Science 244:1288-1292; Capecchi, 1989, Trends in Genet. 5:70-76). The PNS method is more efficient for targeting genes which are not expressed at high levels because the marker gene has its own promoter. Nonhomologous recombinants are selected against by using the Herpes Simplex virus thymidine kinase (HSV-TK) gene and selecting against its nonhomologous insertion with herpes drugs such as gancyclovir (GANC) or FIAU (1-(2-deoxy 2-fluoro-B-D-arabinofluranosyl)-5-iodouracil). By this counter-selection, the percentage of homologous recombinants in the surviving transformants can be increased.

Other methods of producing transgenic mice involve microinjecting the male pronuclei of fertilized eggs. Such methods are well known in the art.

The present invention includes a transgenic, non-human animal in which the animal's genome contains DNA encoding at least a portion of the human Kvβ2.3 subunit where the transgenic non-human animal exhibits altered potassium channel biological activity as compared to a wild-type animal that does not contain a portion of the human Kvβ2.3 subunit. In a preferred embodiment, the non-human transgenic animal is a mouse.

The following non-limiting examples are presented to better illustrate the invention.

### EXAMPLE 1

### Identification of the Human Kvβ2.3 Subunits and cDNA Cloning

First strand cDNA was synthesized at 42° C. for 90 min in a 40 μl reaction containing 2.5 μg human brain poly A+mRNA, 50 mM Tris-HCl, pH 8.3, 8 mM MgCl₂, 3 mM KCl, 2 mM each dNTP, 4 mM DTT, 1.5 μg random hexamer primers, 120 units RNAsin, and 12 units AMV reverse transcriptase.

Kvβ2.3 was then amplified from the cDNA using the polymerase chain reaction and oligonucleotide primers derived from sequence conserved in other (i.e., Kvβ2.1 and Kvβ2.2) members of the Kvβ2 family. Sequences of the primers used were:

| | | |
|---|---|---|
| Forward primer: | 5′-TATAGGACTAGTGCCGCCGCCATGTATCCAGAATCAACG-3′ | (SEQ.ID.NO.:9) |
| Reverse primer: | 5′- ATATATATGCGGCCGCTTAGGATCTGTAGTCC-3′ | (SEQ.ID.NO.:10) |

PCR was carried out in a 100 μl volume consisting of 4 μl of the first strand cDNA, 20 mM Tris-HCl (pH 8.75), 200 μM each dNTP, 10 mM KCl, 10 Mm (NH₄)₂SO₄, 2 mM MgSO₄, 0.1% Triton X-100, 0.1mg/ml BSA, 1 μM of each oligonucleotide primer, and 5 units of Taq plus long polymerase. The reaction was cycled 25 times using the following parameters: 94° C. for 1 min, 56° C. for 2 min, 72° C. for 3 min. cDNAs amplified in this manner were cloned as Spe I-Not I fragments (recognition sites for those 2 enzymes were incorporated into the primers) by standard techniques and then sequenced in their entirety on both strands to determine the nucleotide sequence of Kvβ2.3.

### EXAMPLE 2

### Analysis of Expression of Human Kvβ2.3 Subunit

Northern blot analysis: Northern blots of poly(A+)-RNA isolated from human heart, brain, placenta, lung, liver, skeletal muscle, kidney, pancreas, spleen, thymus, prostate, testes, ovary, small intestine, colon, peripheral blood leukocytes, amygdala, caudate nucleus, corpus callosum, hippocampus, whole brain, substantia nigra, and thalamus were purchased from Clontech (Palo Alto, Calif.) and probed with a ³²P-labeled, oligonucleotide probe derived from the sequence unique to Kvβ2.3. The sequence of the sense strand of the probe was:

5′-GAAGGGGACCCATTTAGTTCCTCCAAGTCAAGGACATTCATCATA-3′ (SEQ.ID.NO.: 11)

The probe was constructed from two overlapping synthetic oligonucleotides that were annealed and filled-in in the presence of all four [³²P]dNTPs and the Klenow fragment of DNA polymerase. The hybridization was carried out in 5×SSPE, 10×Denhardt's solution, 0.5% SDS, 100 μg/ml salmon sperm DNA at 42° C. overnight. Blots were washed stepwise in 2×SSC, 0.05% SDS at 42° C. 3 times for 15-20 minutes, followed by 1×SSC, 0.05% SDS at 50° C. 3-4 times for 15-20 minutes. Hybridization was detected by exposure of the blots to Kodak XAR X-ray film.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various publications are cited herein, the disclosures of which are incorporated by reference in their entireties.

What is claimed is:

1. An isolated DNA comprising nucleotides encoding a human Kvβ2.3 subunit protein.

2. The DNA of claim 1 comprising nucleotides encoding a polypeptide having the amino acid sequence shown in SEQ.ID.NO.: 3.

3. The DNA of claim 1 where the nucleotides comprise positions 1-1146 of SEQ.ID.NO.: 1.

4. An isolated DNA that hybridizes under stringent conditions to the reverse complement of a DNA sequence consisting of positions 1-1146 of SEQ.ID.NO.: 1 where the isolated DNA encodes a potassium channel β subunit protein capable of forming a functional potassium channel with at least one other potassium channel subunit protein.

5. The isolated DNA of claim 4 where the isolated DNA encodes a human Kvβ2 subunit where the isolated DNA comprises the sequence of nucleotides 499-543 of SEQ.ID.NO: 1 and the human Kvβ2 subunit binds to at least one human potassium channel α subunit selected from the group consisting of: Kv1.1, Kv1.2, Kv1.3, Kv1.4, Kv1.5, Kv1.6, Kv1.7, Kv1.8 and Kv1.9.

6. An expression vector comprising the DNA of claim 3.

7. A recombinant host cell comprising the DNA of claim 3.

8. An isolated human Kvβ2.3 subunit protein.

9. The isolated protein of claim 8 having the amino acid sequence shown in SEQ.ID.NO.: 3.

10. The protein of claim 9 containing a single amino acid substitution.

11. The protein of claim 9 containing two or more amino acid substitutions where the amino acid substitutions are conservative substitutions.

12. An isolated human Kvβ2 subunit protein comprising the amino acid sequence GDPFSSSKSRTFIIE (SEQ.ID.NO: 6) where the isolated human Kvβ2 subunit protein is capable of forming a functional potassium channel with at least one other potassium channel subunit protein.

13. An antibody that binds specifically to a human Kvβ2.3 subunit protein.

14. A DNA or RNA oligonucleotide probe comprising at least 10 contiguous nucleotides from SEQ.ID.NO.: 1.

15. A method for identifying substances that modulate the activity of potassium channels containing human Kvβ2.3 subunit protein comprising:
(a) providing cells expressing a potassium channel containing human Kvβ2.3 subunit protein;
(b) loading the cells with ⁸⁶Rb;
(c) measuring the efflux of ⁸⁶Rb from the cells in the presence and in the absence of a substance;
where if the efflux of ⁸⁶Rb from the cells differs in the presence as compared to the absence of the substance, then the substance modulates the activity of potassium channels containing human Kvβ2.3 subunit protein.

16. A method for identifying substances that bind to potassium channels containing human Kvβ2.3 subunit protein comprising:
(a) providing cells expressing a potassium channel containing human Kvβ2.3 subunit protein;
(b) exposing the cells to a substance;
(c) determining the amount of binding of the substance to the cells;
(d) comparing the amount of binding in step (c) to the amount of binding of the substance to control cells where the control cells are substantially identical to the cells of step (a) except that the control cells do not express human Kvβ2.3 subunit protein;
where if the amount of binding in step (c) is greater than the amount of binding of the substance to control cells, then the substance binds to potassium channels containing human Kvβ2.3 subunit protein.

17. A method of identifying substances that bind potassium channels containing human Kvβ2.3 subunit protein comprising:
(a) providing cells expressing potassium channels containing human Kvβ2.3 subunit protein;
(b) exposing the cells to a compound that is known to bind to the potassium channels containing human Kvβ2.3 subunit protein in the presence and in the absence of a substance not known to bind to potassium channels containing human Kvβ2.3 subunit protein;
(c) determining the amount of binding of the compound to the cells in the presence and in the absence of the substance;
where if the amount of binding of the compound in the presence of the substance differs from the amount of binding in the absence of the substance, then the substance binds potassium channels containing human Kvβ2.3 subunit protein.

18. A method of identifying activators or inhibitors of potassium channels containing human Kvβ2.3 subunit protein comprising:
(a) recombinantly expressing human Kvβ2.3 subunit protein or mutant human Kvβ2.3 subunit protein in a host cell so that the recombinantly expressed human Kvβ2.3 subunit protein or mutant human Kvβ2.3 subunit protein is incorporated into functional potassium channels with other potassium channel subunit proteins;
(b) measuring the biological activity of the functional potassium channels of step (a) in the presence and in the absence of a substance;
where a change in the biological activity of the functional potassium channels of step (a) in the presence as compared to the absence of the substance indicates that the substance is an activator or an inhibitor of potassium channels containing human Kvβ2.3 subunit protein.

19. The method of claim 18 where the cells are selected from the group consisting of: Xenopus oocytes, HEK293 cells, and mouse L cells.

20. The method of claim 19 where the biological activity is selected from the group consisting of: the production of a voltage-gated potassium current, the modulation of a voltage-gated potassium current, and the efflux of ⁸⁶Rb from the cells.

21. The method of claim 20 where the modulation of a voltage-gated potassium current is a change in the electrophysiological characteristics of the current that is selected from the group consisting of: a change in gating voltage, a change in activation kinetics, a change in deactivation kinetics, and a change in tail current.

22. A method of identifying inhibitors of potassium channels containing human Kvβ2.3 subunit protein comprising:
(a) expressing human Kvβ2.3 subunit protein in Xenopus oocytes such that potassium channels containing the human Kvβ2.3 subunit protein are formed;
(b) changing the transmembrane potential of the oocytes in the presence and the absence of a substance;
(c) measuring membrane potassium currents following step (b);
where if the membrane potassium currents measured in step (c) are greater in the absence rather than in the presence of the substance, then the substance is an inhibitor of potassium channels containing human Kvβ2.3 subunit protein.

23. A method of identifying activators of potassium channels containing human Kvβ2.3 subunit protein comprising:
(a) providing test cells comprising:
(1) an expression vector that directs the expression of human Kvβ2.3 subunit protein in the cells so that potassium channels containing human Kvβ2.3 subunit protein are formed in the cells;
(2) a first fluorescent dye, where the first dye is bound to the exterior of the plasma membrane of the cells; and
(3) a second fluorescent dye, where the second fluorescent dye is free to move from one face of the plasma membrane of the cells to the other face in response to changes in membrane potential;
(b) exposing the test cells to a substance;
(c) measuring the amount of fluorescence resonance energy transfer (FRET) in the test cells that have been exposed to the substance;
(d) comparing the amount of FRET exhibited by the test cells that have been exposed to the substance with the amount of FRET exhibited by control cells;
where if the amount of FRET exhibited by the test cells is greater than the amount of FRET exhibited by the control cells, the substance is an activator of potassium channels containing human Kvβ2.3 subunit protein;
where the control cells are either (1) cells that are essentially the same as the test cells except that they do not comprise at least one of the items listed at (a) (1)-(3) but have been exposed to the substance; or (2) test cells that have not been exposed to the substance.

24. A method of identifying inhibitors of potassium channels containing human Kvβ2.3 subunit protein comprising:
(a) providing test cells comprising:
(1) an expression vector that directs the expression of human Kvβ2.3 subunit protein in the cells so that potassium channels containing human Kvβ2.3 subunit proteins are formed in the cells;
(2) a first fluorescent dye, where the first dye is bound to the exterior of the plasma membrane of the cells; and
(3) a second fluorescent dye, where the second fluorescent dye is free to move from one face of the plasma membrane of the cells to the other face in response to changes in membrane potential;
(b) exposing the test cells to a substance that is suspected of being an inhibitor of potassium channels containing human Kvβ2.3 subunit protein;
(c) measuring the amount of fluorescence resonance energy transfer (FRET) in the test cells that have been exposed to the substance;
(d) comparing the amount of FRET exhibited by the test cells that have been exposed to the substance with the amount of FRET exhibited by control cells;
where if the amount of FRET exhibited by the test cells is less than the amount of FRET exhibited by the control cells, the substance is an inhibitor of potassium channels containing human Kvβ2.3 subunit protein;
where the control cells are either (1) cells that are essentially the same as the test cells except that they do not comprise at least one of the items listed at (a) (1)-(3) but have been exposed to the substance; or (2) test cells that have not been exposed to the substance.

25. A method of modulating the biological activity of potassium channels containing the Kvβ2.3 subunit in a human host in need of such modulation comprising administering a compound that modulates the activity of a potassium channel containing the Kvβ2.3 subunit where the compound does not also modulate the biological activity of potassium channels that do not contain the Kvβ2.3 subunit.


SEQUENCE LISTING (11 sequences)

SEQ ID NO: 1 - DNA, 1149 bp, Human
atgtatccagaatcaacgacgggctccccggctcggctctcgctgcggcagacgggctcccccgggatgatctacagtactcggtatgggagtcccaaaagacagctccagttttacaggaacctgggcaagtctggcctgcgggtctcctgcctgggacttggaacatgggtgaccttcggaggccagatcaccgatgagatggcagagcagctcatgaccttggcctatgataatggcatcaacctcttcgatacagcagaagtctacgcagccggcaaggctgaagtggtactgggaaacatcattaagaagaaaggatggaggcggtccagcctcgtcatcaccaccaagatcttctggggcggaaaggcggagacggagcggggcctgtccaggaagcacataatcgaaggtctgaaagcttccctggagcgactgcagctggagtacgtggatgtggtgtttgccaaccgcccggaccccaacaccccgatggaaggggacccatttagttcctccaagtcaaggacattcatcatagaagagaccgtccgcgccatgacccacgtcatcaaccaggggatggccatgtactggggcacgtcacgctggagctccatggagatcatggaggcctactccgtggcccggcagttcaacctgaccccgcccatctgcgagcaggctgagtaccacatgttccagcgtgagaaagtggaggtgcagctgccggagctgttccacaagataggagtgggcgccatgacctggtcccctctggcctgtggcattgtttctggcaagtacgacagtggcatcccaccctactcaagagcctccttgaagggctaccagtggctgaaggacaagatcctcagtgaggagggccggcgccagcaagccaagctgaaggagctgcaggccatcgccgagcgcctgggctgcaccctgccccagctggccatagcctggtgcctgaggaatgagggagtcagctccgtgctcctgggggcctccaatgcggaccagctcatggagaacattggggcaatacaggtccttccgaaactgtcatcttccattatccacgagattgatagtattttgggcaataaaccctacagcaaaaaggactacagatcctaa

SEQ ID NO: 2 - DNA, 1149 bp, Human
tacataggtcttagttgctgcccgaggggccgagccgagagcgacgccgtctgcccgagggggccctactagatgtcatgagccataccctcagggttttctgtcgaggtcaaaatgtccttggacccgttcagaccggacgcccagaggacggaccctgaaccttgtacccactggaagcctccggtctagtggctactctaccgtctcgtcgagtactggaaccggatactattaccgtagttggagaagctatgtcgtcttcagatgcgtcggccgttccgacttcaccatgaccctttgtagtaattcttctttcctacctccgccaggtcggagcagtagtggtggttctagaagaccccgcctttccgcctctgcctcgccccggacaggtccttcgtgtattagcttccagactttcgaagggacctcgctgacgtcgacctcatgcacctacaccacaaacggttggcgggcctggggttgtggggctaccttcccctgggtaaatcaaggaggttcagttcctgtaagtagtatcttctctggcaggcgcggtactgggtgcagtagttggtcccctaccggtacatgaccccgtgcagtgcgacctcgaggtacctctagtacctccggatgaggcaccgggccgtcaagttggactggggcgggtagacgctcgtccgactcatggtgtacaaggtcgcactctttcacctccacgtcgacggcctcgacaaggtgttctatcctcacccgcggtactggaccaggggagaccggacaccgtaacaaagaccgttcatgctgtcaccgtagggtgggatgagttctcggaggaacttcccgatggtcaccgacttcctgttctaggagtcactcctcccggccgcggtcgttcggttcgacttcctcgacgtccggtagcggctcgcggacccgacgtgggacggggtcgaccggtatcggaccacggactccttactccctcagtcgaggcacgaggacccccggaggttacgcctggtcgagtacctcttgtaaccccgttatgtccaggaaggctttgacagtagaaggtaataggtgctctaactatcataaaacccgttatttgggatgtcgtttttcctgatgtctaggatt

SEQ ID NO: 3 - protein, 382 aa, Human
MYPESTTGSPARLSLRQTGSPGMIYSTRYGSPKRQLQFYRNLGKSGLRVSCLGLGTWVTFGGQITDEMAEQLMTLAYDNGINLFDTAEVYAAGKAEVVLGNIIKKKGWRRSSLVITTKIFWGGKAETERGLSRKHIIEGLKASLERLQLEYVDVVFANRPDPNTPMEGDPFSSSKSRTFIIEETVRAMTHVINQGMAMYWGTSRWSSMEIMEAYSVARQFNLTPPICEQAEYHMFQREKVEVQLPELFHKIGVGAMTWSPLACGIVSGKYDSGIPPYSRASLKGYQWLKDKILSEEGRRQQAKLKELQAIAERLGCTLPQLAIAWCLRNEGVSSVLLGASNADQLMENIGAIQVLPKLSSSIIHEIDSILGNKPYSKKDYRS

SEQ ID NO: 4 - protein, 367 aa, Human
MYPESTTGSPARLSLRQTGSPGMIYSTRYGSPKRQLQFYRNLGKSGLRVSCLGLGTWVTFGGQITDEMAEQLMTLAYDNGINLFDTAEVYAAGKAEVVLGNIIKKKGWRRSSLVITTKIFWGGKAETERGLSRKHIIEGLKASLERLQLEYVDVVFANRPDPNTPMEETVRAMTHVINQGMAMYWGTSRWSSMEIMEAYSVARQFNLTPPICEQAEYHMFQREKVEVQLPELFHKIGVGAMTWSPLACGIVSGKYDSGIPPYSRASLKGYQWLKDKILSEEGRRQQAKLKELQAIAERLGCTLPQLAIAWCLRNEGVSSVLLGASNADQLMENIGAIQVLPKLSSSIIHEIDSILGNKPYSKKDYRS

SEQ ID NO: 5 - protein, 353 aa, Human
MYPESTTGSPARLSLRQTGSPGMIYRNLGKSGLRVSCLGLGTWVTFGGQITDEMAEQLMTLAYDNGINLFDTAEVYAAGKAEVVLGNIIKKKGWRRSSLVITTKIFWGGKAETERGLSRKHIIEGLKASLERLQLEYVDVVFANRPDPNTPMEETVRAMTHVINQGMAMYWGTSRWSSMEIMEAYSVARQFNLTPPICEQAEYHMFQREKVEVQLPELFHKIGVGAMTWSPLACGIVSGKYDSGIPPYSRASLKGYQWLKDKILSEEGRRQQAKLKELQAIAERLGCTLPQLAIAWCLRNEGVSSVLLGASNADQLMENIGAIQVLPKLSSSIIHEIDSILGNKPYSKKDYRS

SEQ ID NO: 6 - protein, 15 aa, Human
GDPFSSSKSRTFIIE

SEQ ID NO: 7 - DNA, 21 bp, Human
atgtatccagaatcaacgacg

SEQ ID NO: 8 - DNA, 23 bp, Human
ttaggatctgtagtcctttttgc

SEQ ID NO: 9 - DNA, 39 bp, Human
tataggactagtgccgccgccatgtatccagaatcaacg

SEQ ID NO: 10 - DNA, 32 bp, Human
atatatatgcggccgcttaggatctgtagtcc

SEQ ID NO: 11 - DNA, 45 bp, Human
gaaggggacccatttagttcctccaagtcaaggacattcatcata